SEQUENCE LISTING (39 sequences)

SEQ ID NO: 1 - protein, 10 aa, Homo sapiens
LYNPTTYQMD

SEQ ID NO: 2 - protein, 10 aa, Homo sapiens
CRNVSRGREC

SEQ ID NO: 3 - protein, 10 aa, Homo sapiens
CAHYIDGPHC

SEQ ID NO: 4 - protein, 6 aa, Homo sapiens
MGENNT

SEQ ID NO: 5 - protein, 9 aa, Homo sapiens
LVWKYADAG

SEQ ID NO: 6 - protein, 16 aa, Homo sapiens
CGADSYEMEEDGVRKC

SEQ ID NO: 7 - protein, 16 aa, Artificial Sequence
  Synthetically generated peptide
GCGLYNPTTYQMDGCG

SEQ ID NO: 8 - protein, 12 aa, Artificial Sequence
  Synthetically generated peptide
GCRNVSRGRECG

SEQ ID NO: 9 - protein, 12 aa, Artificial Sequence
  Synthetically generated peptide
GCAHYIDGPHCG

SEQ ID NO: 10 - protein, 9 aa, Artificial Sequence
  Synthetically generated peptide
  VARIANT 1: Xaa = 6-aminohexanoic acid
XCMGENNTG

SEQ ID NO: 11 - protein, 13 aa, Artificial Sequence
  Synthetically generated peptide
GCLVWKYADAGCG

SEQ ID NO: 12 - protein, 10 aa, Artificial Sequence
  Synthetically generated peptide
TYNTDTFESM

SEQ ID NO: 13 - protein, 16 aa, Artificial Sequence
  Synthetically generated peptide
GCGTYNTDTFESMGCG

SEQ ID NO: 14 - protein, 7 aa, Artificial Sequence
  Synthetically generated peptide
KPDLSYM

SEQ ID NO: 15 - protein, 10 aa, Artificial Sequence
  Synthetically generated peptide
MPIWKFPDEE

SEQ ID NO: 16 - protein, 11 aa, Artificial Sequence
  Synthetically generated peptide
  VARIANT 1: Xaa = 6-aminohexanoic acid
XCKPDLSYMCG

SEQ ID NO: 17 - protein, 14 aa, Artificial Sequence
  Synthetically generated peptide
GCMPIWKFPDEECG

SEQ ID NO: 18 - protein, 143 aa, Homo sapiens
QVQLVQSEGGVVQPGRSLRLSCAASGFTFSSYAMHWVRQAPGKGLEWVAVISYDGSNKYYADSVKGRFTISRDNSKNTLYLQMNSLRAEDTAVYYCARDEGYYGSGCIDYWGQGTLVTVSSGILGSGGGSGGGGSGGGGLQEF

SEQ ID NO: 19 - protein, 144 aa, Homo sapiens
QVQLVQSEGGVVQPGRSLRLSCAAPGFTFSSYAMHWVRQAPGKGLEWVAVISYDGSNKYYADSVKGRFTISRDNSKNTLYLQMNSLRAEDTAVYYCARDEGYYGSGCIDYWGQGTLVTVSSGILGSGGGGSGGGGSGGGGLQEF

SEQ ID NO: 20 - protein, 150 aa, Homo sapiens
QVQLQQSGPGLVKPSQTLSLTCDISGDSVSSDSAAWNWIRLSPSRGLEWLGRTYYWSKWYTDYAVSVKSRITINPDTSKNQFSLQLNSVTPEDTAVYFCARERYCSSTSCYLDAFDIWGQGTMVTVSGILGSGGGGSGGGGSGGGGLQEF

SEQ ID NO: 21 - protein, 141 aa, Homo sapiens
QVQLQQWGPGLLKPSETLSLTCGVSGGSLSGYYWSWIRQSPGKELEWIGEINQGGSTNYNPSLRSRVTISVDTSKKQLSLKVNSVTASDTAVYYCARETFRGSNCFDSWGQGTLVTVSSGILGSGGGIGGGGSGGGGLQEF

SEQ ID NO: 22 - protein, 260 aa, Homo sapiens
QVQLQQSGPGLVKPSQTLSLTCAISGDSVSSNTAAWNWIRQSPSRGLEWLGRTYYRSKWHNDYAASVKSRITINPDTSKNQFSLQLNSVTPEDTAVYYCRRGVWDFDIWGRGTLVTVSSGILGSGGGGSGGGGSGGGGSQPVLTQSPSVSVAAGETARVTCGGNSFRRRSVCWYQQRPGQAPVLVVYDDSDRPSGIPARFSGSNSGNVATLTISSVEAGDEADYYCQCGIVTMIMWFSAEGQADRPIRNSRQKLISEEDL

SEQ ID NO: 23 - protein, 269 aa, Homo sapiens
QVQLQQSGPGLVKPSQTLSLTCAISGDSVSSNRAAWNWIRQSPSRGLEWLGRTYYRSKWYYDYTLSVQGRITISPDTSRNQVFLQLNYVTPEDTAVYYCARVPQDSSGSRHDAFDFWGQGTMVTVSSGILGSGGGGSGGGGSGGGGSQPVLTQSPSASGTPGQRVTLSCAGSSSDIGSNPVNWYQQLPGTAPKLLMYSNDQRPSGVSDRFSGSKSGTSASLAVSGLQSEDEADYYCAAWDDSLNGWVFGGGTKLTVLSGIQKLISEEDL

SEQ ID NO: 24 - protein, 225 aa, Homo sapiens
QVQLVQSGAEVKKPGASVKISCKASGYTFTRYYMHWVRQAPGQGLEWMGIINPSGGTTSYAEKFQGRVTMTRGTSTRTVYMELSSLGSEDTAVYFCVRDLVDVDDYSGLHAFDIWGQGTMVTVSSGILGSGGGGSGGGGSGGGGSDIVMTQTPATLSVSPGGGATLSCRGQSEYRQQRSLVPAETWPGSQAPHVWCIHEGHWYPSQVQWQCVWDRFHPHHQQTGS

SEQ ID NO: 25 - protein, 259 aa, Homo sapiens
QVQLQQSGPGLVKPSQTLSLTCAISGDTVSSNSAAWHWIRQSPSRGLEWLGRTYYRSKWYNEYAVSVKSRITINPDTSKNQFSLQLDSVTPEDTAVYYCVRDFYVGFAYWGQGTLVTVSSGILGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSFTFGPGTKVDIKSGIQKLISEEDL

SEQ ID NO: 26 - protein, 267 aa, Homo sapiens
EVQLVESGGGLVQPGGSLRLSCAASGFTFSNSAMSWVRQAPGKGLEWVSVIHSGGSTTYYADSVKGRFTISRDNSKNTLYLQMSSLRAEDTAVYYCAKHTSTWWGGEDHYGMDVWGQGTTVTVSSGILGSGGGGSGGGGSGGGGSQSVLTQPASVSGSPGQSVTISCTGTSSDVGSYMFVSWYQQHPGKAPKLMISDVSNRPSGVPDRFSGSKSGNTASLTISGLRAEDEADYYCSSYTSTNTRVFGGGTKLTVLSGIQKLISEEDL

SEQ ID NO: 27 - protein, 257 aa, Homo sapiens
QITLKESGPTLVKPTQTLTLTCTFSGFSLSTNGVGVGWIRQPPGKALEWLALIYWNDDKRYSPSLKRRLTITKDTSKNQVVLTMTNMDPVDTATYYCAHRLLDSGYILPPTFDYWGQGTLVTVSSGILGSGGGGSGGGGSGGGGSEIVLTQSPGTLSLSPGERATLSCRASQSVNSGYLAWYQQKPGQAPRLLISAASSRATGIPDRFSGSGSGTDFTLTISRLEPEDFAVYYCQRYDTSRGFTFGPGTKVDIKSGF

SEQ ID NO: 28 - protein, 251 aa, Homo sapiens
QVQLQQSGPGLVKPSQTLSLTCAISGDSVSSNSAAWNWIRQSPSRGLEWLGRTYYRSKWYNEYAVSVKSRITINPDTSKNHFSLQLNSVTPEDTAVYYCAREIVGTPFDYWGQGTLVTVSSGILGSGGGGSGGGGSGGGGSDIRVTQSPSSLSASVGDRVTITCRTSQSASKYVNWYQQKPGKVPKLLIYAASSLQSGVPSRFSGSGSGTHFTLSISNLQPGDMGTYYCQQGYGTPLTFGGGTKLEIKSGF

SEQ ID NO: 29 - protein, 222 aa, Homo sapiens
QVQLQQSGPGLVKPSQTLSLTCAISGDSVSSNSAAWNWIRQSPSRGLEWLGRTAYRSKWNSDYAASVRSRITINPDTSKNQFSLQLNSVTPDDTAMYYCARSRSSSPIDYWGQGTLVTVSSGILGSGGGGSGGGGSGGGGSEIVLTQSPGTLSLSPGERVTLSCRASQRVSSSYVAWYQQKPGQAPRLLIYGASSRGHWHPRQVQWQWVWDRLHSHHQQTGA

SEQ ID NO: 30 - protein, 270 aa, Homo sapiens
QVQLKQSGPGLVNPSQTLSLTCAISGDSVSSDSAAWNWIRQSPSRGLEWLGRTYYRSKWYNEYTESVKSRIIINPDTSKNQFSLQLNSVTPEDTALYYCARFLRGTFDIWGQGSMVTVSSASTKGPSGILGSGGGGSGGGGSGGGGSNFMLTQPHSVSESPGKTVTISCTGSSGSIATNYVQWYQQRPGSAPTTVIYENNHRPSGVPDRFSGSIDSSSNSASLTISGLKTEDEAGYYCQSSDNDFHAVFGGGTQLTVLSGIQKLISEEDL

SEQ ID NO: 31 - protein, 219 aa, Homo sapiens
QVQLVESEGGVVQPGRSLRLSCAASGFSFSRFAMHWVRQAPGKGLEWVAVISYDGSNKFYADSVKGRFTISRDNSKNTLYLQMDSLRAEDTAVYYCARHYDSSGRDHWGQGTLVTVSSGILGSGGGGSGGGGSGGGGSEIVMTQSPGTLSSSPGERATLSCRASKDVSSQFLAWYQQKPGQAPRLLIYETSTRGHWHPRQVQWQWVWDRLHSHHQQTGA

SEQ ID NO: 32 - protein, 298 aa, Homo sapiens
QVQLQQSGPGLVKPSQTLSLTCAISGDSVSSNSAAWNWIRQSPSRGLEWLGRTAYRSKWNSDYAASVRSRITINPDTSKNQFSLQLNSVTPDDTAMYYCARSRSSSPIDYWGQGTLVTVSSGILGSGGGGSGGGGSGGGGSEIVLTQSPGTLSLSPGERVTLSCRASQRVSSSYVAWYQQKPGQAPRLLIYGASSRTLASQTGSVAVGLGQTSLSPSADWSLKISQCITVSSMIVHLERSARDQAGDQILNSRQSLFLKRLVIASAAASRSDNNSVDVQIDFVPLYFSSYKYIYFHSP

SEQ ID NO: 33 - protein, 264 aa, Homo sapiens
EVQLVESGGGLVQPGGSLRLSCAASGFTFSNSAMSWVRQAPGKGLEWVSVIHSGGSTTYYADSVKGRFTISRDNSKNTLYLQMSSLRAEDTAVYYCAKHTSTWWGGEDHYGMDVWGQGTTVTVSSGILGSGGGGSGGGGSGGGGSQSVLTQPASVSGSPGQSVTISCTGTSSDVGSYMFVSWYQQHPGKAPKLMISDVSNRPSRVPDRFSGSKSGNTASLTISGLWAEDEADYYCSSYTSTNTRVFGEGPSSPSYPESRTKAYF

SEQ ID NO: 34 - protein, 260 aa, Homo sapiens
EVQLVESGGGLVQPGRSLRLSCAASGFSFADYAMHWVRQVPGKGLEWVSSISWNSGIKGYADSVKGRFTISRDNAKNTVYLEVNNIRPEDTALYYCAKVRDPNIEAFDVWGQGTMVTVSSGILGSGGGGSGGGGSGGGGSEIVLTQSPGTLSLSPGERATLSCRASQSVSSSYLGWYQQKPGQAPRLLIYGASSRATGIPDRFSGSGSGTDFALTISRLEPEDFAVYYCQQHDRSSWTFGQGTKVEIKSGIQKLISEEDL

SEQ ID NO: 35 - protein, 262 aa, Homo sapiens
QVQLVESEGRLVKPGGSLRLSCVASGMTFNNAWMSWVRQAPGKGLEWVGRIRSESNGGTTDYAAPGKGRFTISRDDSKNTLYLQMNSLQIEDTAVYYCATDRGYSNSGNYYRHYWGQGTLVTVSSGILGSGGGGSGGGGSGGGGSDIRLTQSPSSLSASVGDRVTITCQASQDISNYLNWYQQKPGKAPKLLIYDNLETGVPPRFSGSGSGTAFTFTISSLQPEDFATYYCQQYDNLPHTFGPGTKVDIKSGIQKLISEEDL

SEQ ID NO: 36 - protein, 170 aa, Homo sapiens
QVQLQESGPGLVKPSETLSLTCTFSGGSIRSSSDYWGWVRQPPGKGLEWIGSISSSGSTYYNPSLKSRVTISVDTSRNQFSLRLSSVTAADTAIYYCARYNHYWYFDLWGRGTLVTVSSGILGSGGGSGGGGSGGGGSKRHSRSLQHSSQRLQETKSTSPAKPAKTLMMI

SEQ ID NO: 37 - protein, 142 aa, Homo sapiens
QVQLQESGPGLVKPSETLSLTCTVSGGSISNSDYYWGWIRQPPGKGLRWIGSINYYGTTYYNPSLKSRVAMSVDTSKNQFSLKLSSVTAADTAVYYCARRGANSWFFDLWGRGTLVTVSSGILGSGGGSGGGGSGGGGLQEF

SEQ ID NO: 38 - protein, 150 aa, Homo sapiens
QITLKESGPTVVKPTQTLTLTCTFSGFSLSTSGVGVGWIRQPPGKALEWLALISWDDDKRYSPSLKSRLTITKDTSKNQVVLTMTNMDPVDTATYFCAHLNNFVDAIELRTGWCFDVWGRGTLVTVSGILGSGGGGSGGGGSGGGVLKLC

SEQ ID NO: 39 - protein, 150 aa, Homo sapiens
QITLKESGPTVVKPTQTLTLTCTFSGFSLSTSGVGVGWIRQPPGKALEWLALISWDDDKRYSPSLKSRLTITKDTSKNQVVLTMTNMDPVDTATYFCAHLINFVDAIELRTGWCFDVWGRGTLVTVSGILGSGGGGSGGGGSGGGVLKLR

(19) United States
(12) Patent Application Publication
(10) Pub. No.: US 2007/0009972 A1
(43) Pub. Date: Jan. 11, 2007

(54) Epidermal growth factor receptor polypeptides and antibodies

(76) Inventors: Ginger Chao, Somerville, MA (US); Mark Olsen, Canyon, TX (US); Alejandro Wolf-Yadlin, Somerville, MA (US); K. Dane Wittrup, Chestnut Hill, MA (US); Douglas A. Lauffenburger, Cambridge, MA (US)

Correspondence Address: FISH & RICHARDSON PC, P.O. BOX 1022, MINNEAPOLIS, MN, 55440-1022, US

(21) Appl. No.: 11/356,385

(22) Filed: Feb. 16, 2006

Related U.S. Application Data
(60) Provisional application No. 60/653,423, filed on Feb. 16, 2005.

(51) Int. Cl. G01N 33/574; C12P 21/06; A61K 38/16; C07K 16/30; C07K 14/47

(52) U.S. Cl. 435/7.23; 435/69.1; 435/326; 435/320.1; 514/12; 530/324; 530/325; 530/326; 530/388.8

(57) ABSTRACT

Antibodies and polypeptides that bind to and/or modulate the activity of receptors in the epidermal growth factor family are provided herein.

### CROSS-REFERENCE TO RELATED APPLICATIONS

This application claims the benefit of priority of U.S. Provisional Application No. 60/653,423, filed on Feb. 16, 2005, the entire contents of which are hereby incorporated by reference.

### FEDERALLY SPONSORED RESEARCH OR DEVELOPMENT

The work described herein was funded, in part, through a grant from the National Cancer Institute (contract number CA96504, project number 6893687). The United States government may, therefore, have certain rights in the invention.

### TECHNICAL FIELD

This invention relates to compositions, including antibodies, that alter the activity of receptors in the epidermal growth factor receptor (EGFR) family. Additional EGFR-related compositions and methods of using such compositions to facilitate wound healing or to slow the rate of unwanted cellular proliferation are also disclosed.

### BACKGROUND

Cellular receptors within the EGFR family include the EGFR (also referred to as HER1 or EebB1), Heregulin2 (also known as HER2 or ErbB2), Heregulin3 (also known as HER3 or ErbB3), and Heregulin 4 (also known as HER4 or ErbB4) (Schlessinger, Cell 110:669-672, 2002). The receptors in this family can be activated by Epidermal Growth Factor (EGF) and Transforming Growth Factor α (TGFα), as well as by ten additional potential ligands (Schlessinger, Cell 110:669-672, 2002). Ligand binding to the receptors induces receptor dimerization and activation of the protein tyrosine kinase activity of the cytoplasmic domains (Schlessinger, Cell 110:669-672, 2002). The members of the EGFR family can either homo- or heterodimerize (Sibilia, et al., Development 130: 4515-4525, 2003).

EGFR family members contain a cytoplasmic region, a single transmembrane region, and an extracellular region with four domains: Domains I, II, III, and IV (or L1, S1, L2, and S2, respectively or alternatively, L1, CR1, L2, and CR2, respectively) (Schlessinger, Cell 110:669-672, 2002; Garrett, et al., Cell 110:763-773, 2002). Studies have indicated that Domains I and III participate in ligand binding, while Domain II is involved in receptor dimerization (Ferguson, et al., Mol. Cell 11:507-517, 2003). One of the functions of Domain IV is autoinhibition of receptor dimerization through the interaction with Domain II (Ferguson, et al., Cell 11:507-517, 2003).

Activation of the tyrosine kinase activity of the EGFR family members leads to the transduction of signals, which result in cell proliferation, differentiation and/or survival (Sibilia, et al., Development 130:4515-4525, 2003). These signals, however, also contribute to tumor resistance to radiation and chemotherapy (Chakravarti et al., Cancer Res. 62:4307-4315, 2002). For example, EGFR amplification and/or truncation has been implicated in the progression of cancers, including but not limited to: colon cancer, non-small cell lung cancer, glioblastoma multiforme, breast cancer, squamous cell cancer, and prostate cancer (Herynk and Radinsky, In vivo 14:587-596, 2000; Bunn and Franklin, Semin. Oncol. 29 (suppl 14):38-44, 2002; Maher et al., Genes Dev. 15:1311-1333, 2001; Navolanic, et al., Int. J. Oncol. 22:237-252, 2003; Spaulding and Spaulding, Semin. Oncol. 29:45-54, 2002).

### SUMMARY

This invention is based, in part, on our discovery of agents that bind to or otherwise associate with and interfere with the function of specific regions of an EGFR (e.g., Domain II, Domain IV, or regions therein). The second and fourth domains can be targeted with, for example, polypeptides that include or that consist of an amino acid sequence of the targeted domain (e.g., Domain II, Domain IV, or regions therein) and/or antibodies that specifically bind to those regions. The targeting agents described herein can modulate (either by stimulating or inhibiting) an activity of an EGFR (e.g., EGFR, HER2, HER3, or HER4, any of which can be human). For example, the agents can modulate (e.g., inhibit) phosphorylation and/or dimerization of an EGFR and, in turn, modulate (e.g., inhibit) one or more of the biological activities of receptors in the EGFR family.

In one aspect, the invention features substantially pure polypeptides that include or that consist of 5-20 (e.g., 6-11, 5-10, 10-15, 8-12, or 15-20) contiguous amino acid residues that are identical to an amino acid sequence present within the second domain or the fourth domain of a receptor in the epidermal growth factor receptor (EGFR) family. Excluded, however, are polypeptides that are naturally occurring receptors in the EGFR family and polypeptides that consist of the sequence CAHYIDGPHC (SEQ ID NO:3), LVWKYADAG (SEQ ID NO:5), or CGADSYEMEEDGVRKC (SEQ ID NO:6). The polypeptides, upon contacting the receptor under physiological conditions, can inhibit an activity of the receptor (e.g., tyrosine kinase activity or receptor dimerization). The polypeptide can also be one that, upon contacting a biological cell that expresses or overexpresses the receptor, inhibits the rate at which the cell proliferates. The receptor may be of a truncated form, and the cell can be a cancer cell. Upon contacting the receptor under physiological conditions, the polypeptide may or may not prevent the receptor from binding its cognate ligand.

In some embodiments, the polypeptide can be cyclic and can include a detectable moiety (e.g., biotin, an enzyme marker, a fluorescent marker, or an epitope tag). As noted below, the polypeptide can include or consist of the sequence LYNPTTYQMD (SEQ ID NO: 1); CRNVSRGREC (SEQ ID NO: 2); MGENNT (SEQ ID NO: 4); or a variant of any of SEQ ID NOs. 1, 2 or 4 in which one, two, or three amino acid residues are mutated (e.g., substituted with another residue or deleted).

The invention also features methods of inhibiting an activity of a receptor in the EGFR family. The method can be carried out by providing a cell that expresses the receptor and contacting the cell, preferably under physiological conditions, with an amount of a polypeptide described herein that is sufficient to inhibit an activity of the receptor. The cell can be one that is maintained in culture or present within a living subject (e.g., a mammal such as a human). The cell can also be one that is proliferating at an undesirable rate such as a cancer cell. The cell can be of any type that expresses an EGFR, and may be a glial cell a squamous cell, or a cell within the lung, breast, colon, or prostate.

The invention also features kits that include one or more of the compositions (e.g., targeting agents) described herein and instructions for diagnostic or therapeutic use.

The invention also features pharmaceutically acceptable compositions that include one or more of the polypeptides described herein and a diluent. The compositions may further include an adjuvant.

Methods of making an antibody useful as a targeting agent are within the scope of the invention and can be carried out by administering to a human or non-human animal: (a) a polypeptide (e.g., a substantially pure polypeptide) that includes a number of contiguous amino acid residues (e.g., 5-20, 5-10, 10-15, 8-12, or 15-20 residues) that are identical to an amino acid sequence present within the second domain or the fourth domain of a receptor in the epidermal growth factor receptor (EGFR) family, with the proviso that the polypeptide is not a naturally occurring receptor in the EGFR family or a polypeptide consisting of the sequence CAHYIDGPHC (SEQ ID NO:3), LVWKYADAG (SEQ ID NO:5), or CGADSYEMEEDGVRKC (SEQ ID NO:6); (b) a pharmaceutically acceptable composition including the polypeptide of (a) and a diluent and/or an adjuvant; (c) a polypeptide including or consisting of the sequence CAHYIDGPHC (SEQ ID NO:3) or LVWKYADAG (SEQ ID NO:5); (d) a pharmaceutically acceptable composition including the sequence CAHYIDGPHC (SEQ ID NO:3) or LVWKYADAG (SEQ ID NO:5) and a diluent; or (e) a pharmaceutically acceptable composition including the sequence CAHYIDGPHC (SEQ ID NO:3) or LVWKYADAG (SEQ ID NO:5), a diluent, and an adjuvant. Antibodies subsequently produced can be isolated, cloned, and expressed in a cell (e.g., a cell of a cell line). The antibodies can be human or humanized.

The isolated antibodies or antigen-binding portions thereof can selectively bind to a polypeptide consisting of 5-20 contiguous amino acid residues that are identical to an amino acid sequence present within the second domain or the fourth domain of an EGFR, with the proviso that the antibody is not the antibody designated 806 (described in Jungbluth et al., Proc. Natl. Acad. Sci. USA 100(2):639-644, 2003) or an antibody that specifically binds a polypeptide consisting of CGADSYEMEEDGVRKC (SEQ ID NO:6). For example, the targeting agent can be an isolated antibody, an antigen-binding portion thereof, or a modified antibody (e.g.,, a humanized or single chain antibody) that selectively binds to a polypeptide represented by SEQ ID NO:1, SEQ ID NO:2, SEQ ID NO:3, SEQ ID NO:4, or SEQ ID NO:5. The antibody per se, or an antigen-binding portion thereof, can include a sequence that is at least 80% identical (e.g., at least 85%, 90%, 95%, 98%, or 99% identical) to SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; or SEQ ID NO:39.

In various embodiments, the agent includes one or more antigen-binding portions of SEQ ID NO:18; SEQ ID NO:19; SEQ ID NO:20; SEQ ID NO:21; SEQ ID NO:22; SEQ ID NO:23; or SEQ ID NO:24; SEQ ID NO:25; SEQ ID NO:26; SEQ ID NO:27; SEQ ID NO:28; SEQ ID NO:29; SEQ ID NO:30; SEQ ID NO:3 1; SEQ ID NO:32; SEQ ID NO:33; SEQ ID NO:34; SEQ ID NO:35; SEQ ID NO:36; SEQ ID NO:37; SEQ ID NO:38; or SEQ ID NO:39; or a sequence at least 80% identical (e.g., at least 85%, 90%, 95%, 98%, or 99% identical) to an antigen binding portion of one of these sequences. For example, the agent can include a fragment comprising one or more complementarity determining regions from one of these sequences.

The antibody can be a full-length antibody or a single chain antibody, and the antigen-binding portion can be an Fab or F(ab′)₂ fragment. Any of the antibodies can include a label, a toxin, or a therapeutic agent (e.g., an activator of apoptosis). Upon contacting a receptor within the EGFR family, preferably under physiological conditions, the antibody can inhibit an activity of the receptor (e.g., tyrosine kinase activity or receptor dimerization). Moreover, upon contacting a biological cell that expresses or overexpresses the receptor or expresses a mutant (e.g., truncated) form of the receptor, the antibody can inhibit the rate at which the cell proliferates. The cell can be a cancer cell. As with the polypeptide targeting agents described above, the antibody-based targeting agents may or may not prevent the receptor from binding its cognate ligand.

The invention also features methods of inhibiting an activity of a receptor in the EGFR family. The methods can be carried out by providing a cell that expresses the receptor and contacting the cell, preferably under physiological conditions, with an amount of an isolated antibody described herein that is sufficient to inhibit an activity of the receptor. As with the polypeptide-based targeting agents, the cell can be one that is maintained in cell or tissue culture or one that is present within a living subject (e.g., a mammal such as a human). The cell contacted can be an apparently healthy cell or a cancerous cell and, in either event, can be a glial cell, a squamous cell, or a cell within the lung, breast, colon, or prostate. The method can also include a step in which the cell is contacted with a second anti-cancer agent (e.g., a chemotherapeutic agent or a radioactive agent). Whether the targeting agent is a polypeptide or antibody, it can be administered to a cell (e.g., in the context of treating a patient who has a cancer associated with misexpression of an EGFR) in combination with an antibody, such as a previously validated antibody (e.g., Cetuximab (ERBITUX) or other chemotherapeutic agent. The polypeptide(s), antibody (or antibodies) and the therapeutic agent may be combined in a single formulation, and such formulations are within the scope of the present invention. The “second” antibody can be one that inhibits interaction between the growth factor (EGF) and an EGFR.

Other methods include methods of identifying an antibody, or an antigen binding portion thereof, that specifically binds to Domain II or Domain IV of a receptor within the EGFR family. The methods can be carried out by (a) providing a library of antibodies (e.g., a nonimmune human scFv library), or antigen binding portions thereof, (b) contacting members of the library with a polypeptide comprising Domain II or Domain IV of the receptor, under conditions that allow the antibodies, or antigen binding portions thereof, to specifically bind the polypeptide; and (c) identifying a selected antibody, or antigen-binding portion thereof, that binds to the polypeptide. The library of antibodies, or antigen binding portions thereof, can include human antibodies or antigen binding portions thereof. The antibodies can be single chain antibodies. Any of the antibodies can be expressed on the surfaces of yeast cells, and the methods can further include a step of affinity-maturing the selected antibody, or antigen binding portion thereof, in vitro. The antibodies can be modified by random mutagenesis and screened or rescreened for an ability to bind an EGFR and/or inhibit an activity of the EGFR. Antibodies, or antigen-binding portions thereof, made by such a method are also within the scope of the invention.

The details of one or more embodiments of the invention are set forth in the accompanying drawings and the description below. Other features, objects, and advantages of the invention will be apparent from the description and drawings, and from the claims. All cited patents, patent applications, and references (including references to public sequence database entries) are incorporated by reference in their entireties for all purposes. U.S. Provisional Application No. 60/653,423, filed on Feb. 16, 2005, is incorporated by reference in its entirety for all purposes.

### DESCRIPTION OF DRAWINGS

FIG. 1 is a bar graph depicting activity of O2 and O4 antibodies.

FIG. 2 is a photograph of a Western blot in which samples from cells incubated in the presence or absence of O2 antibody (0.1 or 1 μM) were immunoprecipitated for EGFR and blotted for HER2.

FIG. 3 is a graph depicting levels of phosphorylation of tyrosine 1173 of EGFR relative to total EGFR.

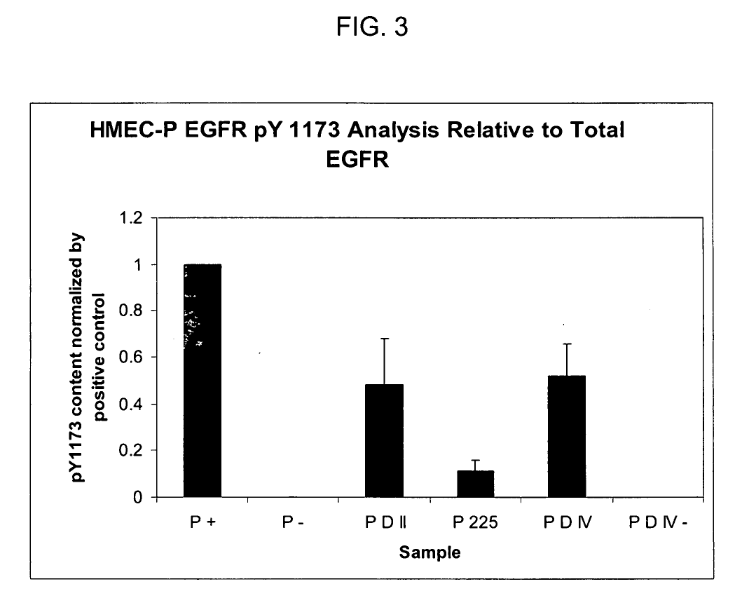

FIG. 4 is a graph depicting levels of phosphorylation of tyrosine 1173 of EGFR relative to total EGFR.

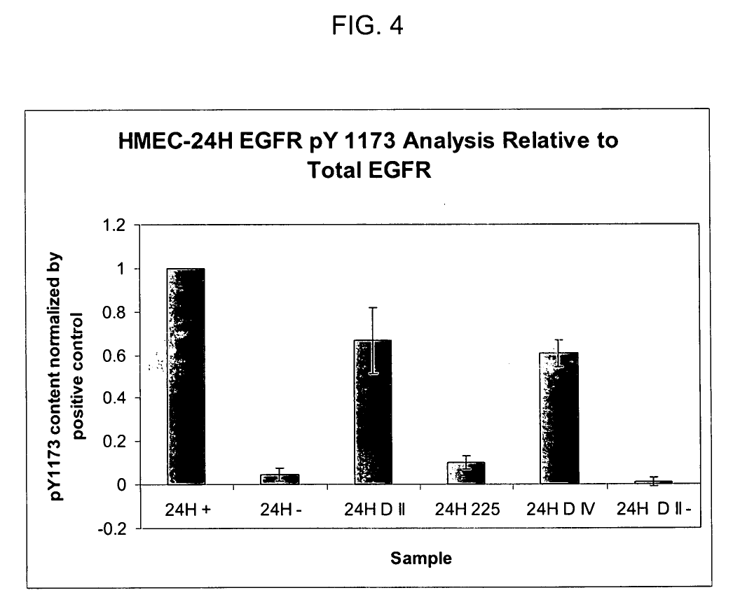

FIG. 5 is a graph depicting levels of phosphorylation of tyrosine 1248 of HER2 relative to total EGFR.

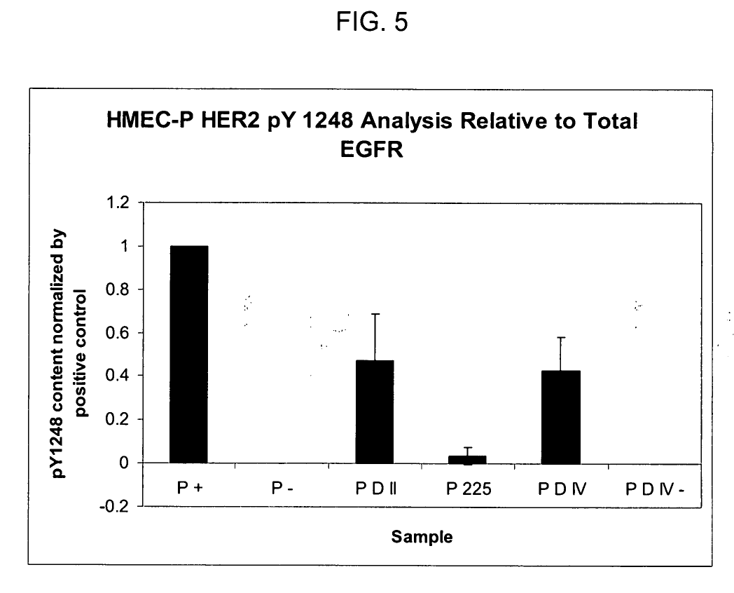

FIG. 6 is a graph depicting levels of phosphorylation of tyrosine 1248 of HER2 relative to total EGFR.

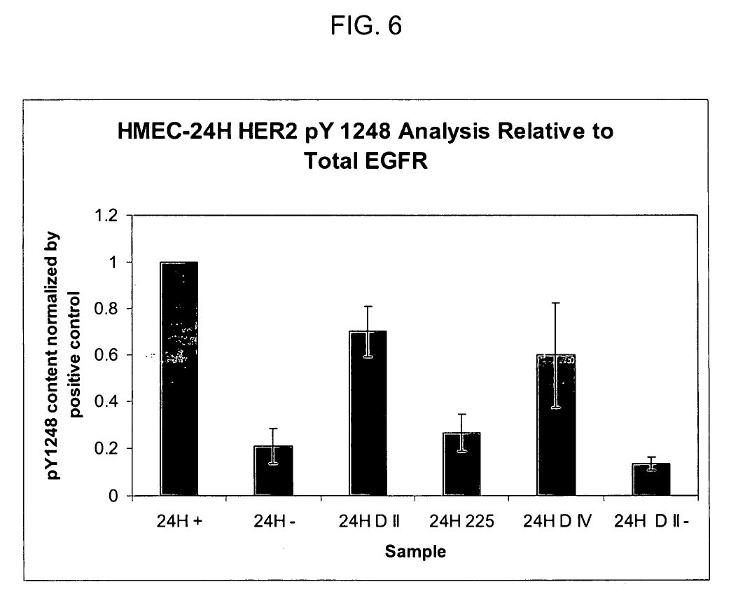

### DETAILED DESCRIPTION

Polypeptides and antibodies that target specific regions within receptors of the EGFR family (e.g., a human EGFR) can be used to modulate one or more of those receptors' activities and can, therefore, be used to diagnose, treat, or prevent disorders associated with abnormal or undesirable receptor activity. Our studies indicate that polypeptides and antibodies that target certain regions within an EGFR can: (1) inhibit unregulated cellular proliferation in cells that abnormally express EGFRs; (2) sensitize tumors to the effects of radiation and chemotherapy; and (3) serve as diagnostic agents (for a review of EGFR expression and measurement in solid tumors see Spaulding and Spaulding, Semin. Oncol. 29:45-54, 2002). Accordingly, the invention provides novel polypeptides and antibodies that target amino acid sequences within receptors in the EGFR family. We may refer to either the polypeptides or antibodies as targeting agents. The invention also features kits and other compositions (e.g., pharmaceutical preparations) that include the targeting agents and various methods for using the targeting agents in diagnostic and therapeutic regimes.

The human EGFR is a transmembrane polypeptide containing 1186 amino acids, 621 of which are located extracellularly. The amino acid sequence of a human EGFR, including a signal sequence, can be found under GenBank® Accession No. CAA25240. A nucleotide sequence encoding that polypeptide can also be found in GenBank® (see GenBank® Accession No. X00588). Domain I of the human EGFR includes amino acids 29-146; Domain II includes amino acids 147-332; Domain III includes amino acids 333-460; and Domain IV includes amino acids 461-600 (these numbers refer to an amino acid sequence lacking the signal peptide) (Lax et al., Mol. Cell. Biol. 8:1831-1834, 1988). Cysteine-rich Domains II and IV contain a series of protruding disulfide-bonded loops (Ferguson et al., Cell 11:507-517, 2003).

Others have generated antibodies against EGFRs and attempted to identify EGFR antagonists by screening for antibodies that inhibit interaction between the receptors and their ligand binding partners (e.g., EGF). However, for truncated EGFR variants, such as EGFRvIII, ligand binding does not trigger EGFR phosphorylation. Thus, it was our belief that where truncated variants are involved, there is a limited benefit to identifying antibodies that interfere with ligand binding. By the methods described herein, we can identify agents that inhibit dimerization or phosphorylation of a receptor in the EGFR family, but that do not target a region of the receptor that participates in ligand binding. More specifically, we have found that agents that target the cysteine-rich regions of Domain II and/or Domain IV can modulate the activity of the receptors. These domains contain disulfide-bonded loops that may be important for dimerization and activation of the receptors. Thus, the inhibitory targeting agents described herein can, upon binding a receptor in the EGFR family, inhibit dimerization, phosphorylation, or any other receptor activity that inhibits receptor activation and thereby inhibit unwanted downstream events mediated by receptor activation (e.g., unwanted cellular proliferation). Similarly, the stimulatory targeting agents can, upon binding a receptor in the EGFR family, facilitate dimerization, phosphorylation, or another receptor activity that stimulates receptor activation and thereby promotes desirable EGFR-mediated processes (e.g., wound healing).

Other antibodies, generated by monoclonal antibody technology, and/or based upon polypeptide sequences generated by aberrant cancer cell DNA splicing events, are able to bind to EGFR or EGFRvIII, but have no impact upon the ability of EGFR or EGFRvIII to trigger the cascade of phosphorylation events involved with cellular proliferation (Ohman, et al., Tumour Biol. 23:61-69, 2002).

Polypeptide-based Targeting Agents

The invention features polypeptides that include amino acid sequences that target Domain II or Domain IV of a receptor in the EGFR family (e.g., EGFR, HER2, HER3, or HER4). Inhibitory targeting agents can bind to the specified domains and inhibit an event associated with receptor activation (e.g., autophosphorylation or dimerization). The sequence of the targeting agent can be, or can include, a sequence found within the second and/or fourth domains of a receptor in the EGFR family. For example, the polypeptides can include between five and 20 amino acid residues (e.g., 6, 7, 8, 9, 10, or 11 amino acid residues) of a Domain II or Domain IV amino acid sequence. For example, the polypeptide can include 5-9 consecutive amino acid residues of the Domain II sequence LYNPTTQMD (SEQ ID NO:1); 5-10 consecutive amino acid residues of the Domain IV sequence CRNVSRGREC (SEQ ID NO:2); or 5-6 consecutive amino acid residues of the Domain IV sequence MGENNT (SEQ ID NO:4) or mutants or variants thereof, as described further below.

The polypeptides can be substantially purified and can modulate one or more biological activities of a receptor in which an EGFR is present, either as a heterodimer or homodimer, as well as variant forms of these receptors (e.g., truncated forms or forms containing other deletions or mutations). The variant form may be one associated with tumor formation or progression.

The sequences derived from Domain II or Domain IV of an EGFR may be flanked by additional amino acid residues, which may or may not alter the function of the EGFR-derived portion of the molecule. Suitable flanking sequences include one or more (e.g., 1-20) glycine residues. The polypeptides may be cyclic. For example, the polypeptides can include cysteine residues that form disulfide bonds. These cysteine residues may be found in the Domain II or Domain IV amino acid sequence or may be added to either end of the Domain II or Domain IV sequence at a distance appropriate for cyclization. The cyclic form of a given polypeptide may modulate a function of an EGFR where the non-cyclic form does not. In some embodiments, the polypeptides described herein include amino acid residues that differ from those found in the second and/or fourth domains of a receptor in the EGFR family (or an EGFR family member). With respect to a wild type sequence, the polypeptide can include one or more mutations. For example, the polypeptide can include one or more deletions, additions, and/or substitutions of, for example, 1, 2, 3, 4, or 5 amino acid residues relative to a wild-type sequence. These polypeptides can also be described as exhibiting a certain degree of identity to a wild type sequence. For example, a mutant polypeptide, or the portion thereof corresponding to Domain II or Domain IV, can be at least 80% (e.g., 85, 86, 87, 88, or 89%), 90%, 95% or more (e.g., 96, 97, 98, or 99%) identical to the corresponding wild type sequence.

We use the terms “identity” and “identical” in connection with protein or DNA sequences to refer to the subunit sequence identity between two molecules. When a subunit position in both of the molecules is occupied by the same monomeric subunit (i.e., the same amino acid residue or nucleotide), then the molecules are identical at that position. If necessary or desired, sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Where a mutant polypeptide differs from a reference sequence (e.g., a portion of a wild type EGFR), the differences may constitute a substitution of one or more amino acid residues. The substitution can be, but is not necessarily, a “conservative” substitution. Examples of conservative substitutions include substitutions within the following groups: glycine, alanine; valine, isoleucine, leucine; aspartic acid, glutamic acid; asparagine, glutamine; serine, threonine; lysine, arginine; and phenylalanine, tyrosine.

The substitutions may also be of an amino acid residue that does not occur in nature (e.g., a beta-amino acid (e.g., β-alanine or norleucine). Polypeptides can also differ from a corresponding wild type sequence by virtue of the manner in which they are post-translationally modified (e.g., glycosylated).

Functionally, a polypeptide including a mutant sequence can modulate receptor activity to a useful extent (e.g., to a clinically beneficial extent). For example, where the mutant polypeptide acts as an inhibitory targeting agent, the polypeptide would inhibit one or more of the receptor's activities to such an extent that it would be useful in treating or preventing a condition associated with abnormal receptor activation (e.g., an EGFR-associated cancer). Similarly, where the mutant polypeptide acts as a stimulatory targeting agent, the polypeptide would stimulate one or more of the receptor's activities. For example, the polypeptide may stimulate receptor activity to an extent that speeds wound healing. In either event (whether inhibition or stimulation is desired), the mutant polypeptide can modulate the receptors' activities by modulating receptor phosphorylation or dimerization.

Polypeptides can be readily assessed for their ability to modulate receptor activity in vivo or in cell culture, and they may be assessed in the presence of ligands. For example, a given polypeptide can be assessed for its ability to stimulate or inhibit EGFR activity (e.g., phosphorylation) in response to EGF binding. Receptor activity can be assessed in numerous ways using methods known to those of ordinary skill in the art. For example, phosphorylation can be assessed using the HMEC-based assays described herein (HMEC represents Human Mammary Epithelial Cells). Thus, receptor activity can be assessed using cellular activity (e.g., the rate of proliferation) as an endpoint. As noted above, the polypeptides can be assessed in response to ligand binding and/or in response to proliferation mediated by ligand-independent forms of a receptor in the EGFR family.

Where a polypeptide of the invention exists in nature, it may be “substantially pure,” meaning that it has been separated to some extent from other components with which it is naturally associated. For example, a polypeptide of interest can constitute at least 60% by weight (dry weight) of a composition. For example, a composition that includes the polypeptide can be at least 65%, 70%, 75%, 80%, 85%, 90%, 95%, or 99%, by weight, the polypeptide of interest. Purity can be measured by any appropriate standard method (e.g., column chromatography, polyacrylamide gel electrophoresis, or HPLC analysis). We expect that many of the polypeptides of the invention will not exist in nature. For those polypeptides, while it may be desirable to use them in relatively pure form, it is not necessary to specify that they are “pure” or “substantially pure” in order to distinguish them from naturally occurring polypeptides.

The polypeptides can be generated synthetically or produced by expression of a recombinant nucleic acid molecule. The techniques required to make the polypeptides are routine in the art, and they can be performed without resort to undue experimentation by one of ordinary skill in the art. For example, and-as noted, the polypeptides can be chemically synthesized or produced via expression of nucleic acid molecules generated by recombinant molecular biological techniques. If desired, a combination of these techniques may be used. Modification of the polypeptides (e.g., biotinylation or cyclization) is also routine, and any of the polypeptides described herein can be modified.

The polypeptides can also be a part of a fusion or chimeric polypeptide, which would include a portion of Domain II or Domain IV of a receptor in the EGFR family and a heterologous polypeptide (i.e., a polypeptide that is not derived from Domain II or Domain IV) (see U.S. Pat. No. 6,451,308). The heterologous polypeptide can, for example, be included to increase the circulating half-life of the chimeric polypeptide in vivo. Thus, the heterologous polypeptide can be a serum albumin, such as human serum albumin, or the Fc region of an immunoglobulin (e.g., an IgG). The Fc region can include a mutation that inhibits complement fixation and Fc receptor binding, or it may be lytic (i.e., able to bind complement or to lyse cells via another mechanism, such as antibody-dependent complement lysis (ADCC)). Lytic IgG Fc can target cells for antibody dependent cellular cytotoxicity or complement directed cytolysis (CDC). Appropriate mutations for human IgG are known (see, e.g., Morrison et al., The Immunologist 2:119-124, 1994; and Brekke et al., The Immunologist 2:125, 1994).

The Fc region can also be a naturally occurring or synthetic polypeptide that is homologous to an immunoglobulin (e.g., IgG) C-terminal domain produced by digestion with papain. IgG Fc has a molecular weight of approximately 50 kDa. The peptides described herein can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the peptides. Native activity is not necessary or desired in all cases.

In other embodiments, the polypeptide can include an amino acid sequence derived from Domain II or Domain IV of a receptor in the EGFR family member and a moiety that functions as an antigenic tag. The moiety can be, for example, biotin or a polypeptide such as a FLAG sequence that serves as an epitope tag. FLAG sequences are recognized by biotinylated, highly specific, anti-FLAG antibodies (see Blanar et al., Science 256:1014, 1992; LeClair et al., Proc. Natl. Acad. Sci. USA 89:8145, 1992). The tag can be used to detect a receptor to which the polypeptide is bound and/or assist in protein purification.

In addition to, or in place of, the heterologous polypeptides described above, a polypeptide can include a marker or reporter such as β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacz (encoding β-galactosidase), and xanthine guanine phosphoribosyltransferase (XGPRT). As with many of the standard procedures associated with the practice of the invention, one of ordinary skill in the art will be aware of (or will recognize) additional useful agents that can serve the function of a marker or reporter.

In other embodiments, one can generate a polypeptide including an amino acid sequence derived from Domain II or Domain IV of a receptor in the EGFR family and an antibody or antigen-binding portion thereof. The antibody or antigen-binding component can serve as a targeting moiety, e.g., to localize the chimeric protein to a particular subset of cells or bodily location, or target molecule. Methods of generating chimeric polypeptides that include an antibody or antigen-binding portion thereof are described in, for example, U.S. Pat. No. 6,617,135.

While we expect the polypeptides used most often to practice the methods of the invention will be those in which the EGFR sequence is human, the invention is not so limited. The portion of Domain II or Domain IV can be that expressed by any animal that expresses EGFRs. For example, the sequence can be a mammalian sequence (e.g. that of a mouse, rat, guinea pig, cow, sheep, horse, pig, rabbit, monkey, baboon, dog, or cat).

In the preceding paragraphs, we tended to use the term “polypeptide,” but we may use any of the terms “protein,” “peptide,” or “polypeptide” to refer to polymers of amino acid residues, regardless of the length of the polymer or whether or not it includes post-translational modifications (e.g., glycosylation or phosphorylation). The amino acid residues may be naturally occurring or synthetic, and the sequence may or may not be found in nature. We may note that a particular protein, peptide, or polypeptide “includes” a certain sequence or a certain number of amino acid residues. In that event, the protein, peptide, or polypeptide may include only the specified sequence or number of amino acid residues, or may include one or more additional residues. For ease of reading, we may refer to “an EGFR.” It is to be understood that “an EGFR” can be any of the receptors within the EGFR family.

Nucleic Acid Molecules Encoding Polypeptide Targeting Agents

Nucleic acid molecules that encode a polypeptide described herein are also within the scope of the present invention. Where the nucleic acid encodes a portion of a receptor within the EGFR family, the nucleic acid sequence can be identical to a sequence found in nature (e.g., a portion of the gene encoding human EGFR or any other species of animal described herein) or it may be a degenerate variant thereof.

In keeping with the polypeptide targeting agents, the nucleic acid molecules encode polypeptides that include less than a complete EGFR (i.e., polypeptides that include some or all of Domain II and/or Domain IV, but not the adjoining domains). For example, the nucleic acid molecules can encode 5-20 contiguous amino acid residues found within Domain II or Domain IV (e.g., 6, 7, 8, 9, 10, or 11 amino acid residues). The Domain II- or Domain IV-derived residues can be flanked by additional residues, and those additional residues can be encoded by the nucleic acid molecules. For example, the nucleic acid molecules may include codons that encode one or more (e.g., two) cysteine residues that facilitate the formation of cyclic polypeptides.

The nucleic acid molecules can also encode polypeptide targeting agents that include mutations (e.g., as described above) and/or that are fused (directly or indirectly) to heterologous polypeptides that extend the circulating half-life of the targeting agent or serve as antigenic tags, markers, or reporters.

The nucleic acid molecules may also include non-coding sequences that lie upstream or downstream from a coding sequence (e.g., vector sequences or regulatory elements). In addition, and particularly where the polypeptides are to be secreted by a cell, the nucleic acid molecules can also encode a leader sequence (e.g., an immunoglobulin leader sequence). The nucleic acid molecules can also include sequences that facilitate homologous recombination (e.g., sequences that facilitate incorporation into the genome of a heterologous cell or into the genome of a homologous cell at a position other than the natural chromosomal location). Such cells are also within the scope of the present invention.

As noted, the nucleic acid molecules described herein can also include vector sequences that direct the expression of the targeting agents in, for example, a cell that has been transduced with the vector. Accordingly, expression vectors, such as plasmids, that include a nucleic acid molecule encoding a polypeptide that includes a portion of the second and/or fourth domains of an EGFR and cells transfected with those vectors are within the scope of the invention.

Vectors suitable for use in the present invention include T7-based vectors, which can be used to transduce bacteria (see, for example, Rosenberg et al., Gene 56:125, 1987), the pMSXND expression vector and similar vectors that can be used to transduce mammalian cells (Lee and Nathans, J. Biol. Chem. 263:3521, 1988), and baculovirus-derived vectors (for example the expression vector pBacPAK9 from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Other suitable promoters and additional regulatory elements (e.g., an enhancer) will be known to one of ordinary skill in the art.

Where the transduced cell is of a higher eukaryote, tissue-specific and cell type-specific promoters can be included. These promoters are so named for their ability to direct expression of a nucleic acid molecule in a given tissue or cell type within the body.

In addition to sequences that facilitate transcription of the inserted nucleic acid sequence, vectors can contain origins of replication, and other sequences (e.g., sequences that encode a selectable marker). For example, the neomycin-resistance (neo^{r}) gene imparts G418 resistance to cells in which it is expressed, and thus permits phenotypic selection of the transfected cells. One of ordinary skill in the art can readily determine whether a given regulatory element or selectable marker is suitable for use in a particular context. Other sequences that can be incorporated may facilitate insertion of the sequence encoding the targeting agent into the genome of a host cell. Of course, the heterologous sequences that may be contained within the polypeptide targeting agents can be encoded by nucleic acid sequences included within any given vector.

Viral vectors encompassed by the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a polypeptide from Domain II or Domain IV of an EGFR are also features of the invention. A cell of the invention is a transfected cell; that is, a cell into which a nucleic acid molecule described herein has been introduced by means of recombinant DNA techniques. The progeny of such cells are also considered within the scope of the invention. The cells may be maintained in tissue culture or frozen with a cryoprotectant agent.

The precise components of the expression system are not critical. For example, a polypeptide can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. One of ordinary skill in the art is able to make such a determination. Furthermore, if guidance is required in selecting an expression system, skilled artisans may consult Ausubel et al. (Current Protocols in Molecular Biology, John Wiley and Sons, New York, N.Y., 1993) or Pouwels et al. (Cloning Vectors: A Laboratory Manual, 1985 Suppl. 1987).

The expressed polypeptides can be purified from the expression system using routine biochemical procedures, and can be used, for example, as diagnostic and therapeutic agents, as described herein.

Antibody-based Targeting Agents

Also provided herein are antibodies that specifically bind to a receptor within the EGFR family and modulate the receptor's function. These targeting agents can specifically bind to the second or fourth domains.

The antibodies can be raised by immunizing animals with a polypeptide described herein, including a cyclic polypeptide. The antibodies can be identified by screening an antibody library (e.g., using yeast display).

The antibodies can assume various configurations. For example, they can be a tetramer (e.g., an antibody having two heavy chains and two light chains) or a single-chain antibody. Accordingly, the antibodies provided herein include polypeptides that may have one or two heavy chain variable regions, and one or two light chain variable regions. The variable heavy chain (VHC) and variable light chain (VLC) regions can be further subdivided into regions of hypervariability, termed “complementarity determining regions” (CDRs), interspersed with regions that are more conserved, termed “framework regions” (FRs). The extent of the FRs and CDRs has been defined (see, Kabat et al. Sequences of Proteins of Immunological Interest, Fifth Edition, U.S. Department of Health and Human Services, NIH Publication No. 91-3242, 1991, and Chothia et al., J. Mol. Biol. 196:901-917, 1987, which are incorporated herein by reference). Where an antibody described herein includes one or more VHCs and/or one or more VLCs, each VHC and VLC can be composed of three CDRs and four FRs, arranged from amino-terminus to carboxy-terminus in the following order: FR1, CDR1, FR2, CDR2, FR3, CDR3, FR4.

The VHC or VLC of an antibody provided herein can further include all or part of a heavy or light chain constant region. In one embodiment, the antibody is a tetramer of two heavy immunoglobulin chains and two light immunoglobulin chains; the heavy and light immunoglobulin chains can be inter-connected by, for example, disulfide bonds. The heavy chain constant region includes three domains: CH1, CH2 and CH3. The light chain constant region is comprised of one domain: CL. The variable regions of the heavy and light chains contain a binding domain that interacts with an antigen. The constant regions of the antibodies typically mediate the binding of the antibody to host tissues or factors, including various cells of the immune system (e.g., effector cells) and the first component (Clq) of the classical complement system. The antibody can be an intact immunoglobulin of any of types IgA, IgG, IgE, IgD, IgM (as well as subtypes thereof (e.g., IgG₁, IgG₂, IgG₃, and IgG₄)), and the light chains of the immunoglobulin may be of types kappa or lambda.

As antibodies may also be referred to as “immunoglobulins” (proteins consisting of one or more polypeptides substantially encoded by immunoglobulin genes), the anti-EGFR antibodies described herein may also be referred to as anti-EGFR immunoglobulins, and may contain sequences encoded by one or more of the human immunoglobulin genes. The recognized human immunoglobulin genes include the kappa, lambda, alpha (IgA₁ and IgA₂), gamma (IgG₁, IgG₂, IgG₃, IgG₄), delta, epsilon, and mu constant region genes, as well as the myriad immunoglobulin variable region genes. Full-length immunoglobulin light chains (about 25 kDa and 214 amino acids) are encoded by a variable region gene at the NH₂-terminus (about 110 amino acids) and a kappa or lambda constant region gene at the COOH-terminus. Full-length immunoglobulin heavy chains (about 50 kDa and 446 amino acids), are similarly encoded by a variable region gene (about 116 amino acids) and one of the other aforementioned constant region genes, e.g., gamma (encoding about 330 amino acids). The antibodies or immunoglobulins of the present invention may include CDRs (which are described further herein) from a human or non-human source. The framework of the immunoglobulin can be human, humanized, or non-human, e.g., a murine framework modified to decrease antigenicity in humans, or a synthetic framework, e.g., a consensus sequence.

The term “antigen-binding portion” of an antibody (or similar terms such as “antibody portion,” or “antibody or a portion thereof”), as used herein, refers to a portion of an antibody that specifically binds to an EGFR, e.g., a molecule in which one or more of the immunoglobulin chains is not full length, but which specifically binds to an EGFR. Examples of binding portions encompassed within the term “antigen-binding portion” of an antibody include (i) a Fab fragment, a monovalent fragment consisting of the VLC, VHC, CL and CH1 domains; (ii) a F(ab′)₂ fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment (Ward et al., Nature 341:544-546, 1989), which consists of a VHC domain; and (vi) an isolated complementarity determining region (CDR) having sufficient framework to specifically bind, e.g., an antigen binding portion of a variable region. An antigen-binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFv); see e.g., Bird et al. Science 242:423-426, 1988; and Huston et al. Proc. Natl. Acad. Sci. USA 85:5879-5883, 1988). Such single chain antibodies are also intended to be encompassed within the term “antigen-binding portion” of an antibody. These antibody portions are obtained using conventional techniques known to those with skill in the art, and the portions are screened for utility in the same manner as are intact antibodies. A Fab fragment can result from cleavage of a tetrameric antibody with papain; Fab′ and F(ab′)₂ fragments can be generated by cleavage with pepsin.

Human single-chain antibodies specific for EGFR were produced as described herein. In a particular embodiment, we provide antibodies that have specificity for EGFR, and bind to an epitope bound by an antibody described herein. Antibodies that bind an epitope that overlaps with an epitope bound by an antibody described herein can be identified by their ability to compete for binding to EGFR with an EGFR Domain II or Domain IV peptide (e.g., to cells bearing EGFR, such as EGFR transfectants, or EGFR-bearing tumor cells).

The antibodies described herein can be polyclonal or monoclonal. These antibodies and antigen binding portions thereof are useful in therapeutic compositions and regimens, diagnostic compositions and regimens, and in assays requiring an agent that can identify or inhibit an EGFR. The present invention encompasses an antibody or antigen binding portion thereof for use in therapy (including prophylaxis) or diagnosis (e.g., of particular diseases or conditions such as cancers), and use of such antibodies or antigen binding portions thereof for the manufacture of a medicament for use in treatment of diseases or conditions as described herein.

Antibodies that specifically bind to particular domains within an EGFR (e.g., the second or fourth domain) can be identified by expressing recombinant antibodies in a library and selecting members of the library that bind the given EGFR. The affinity of the selected antibodies for an EGFR can be further enhanced by affinity-maturing these antibodies. Maturation can be achieved using PCR mutagenesis, chain shuffling, or CDR shuffling techniques in conjunction with one or more cycles of screening.

Other methods can also be used to generate anti-EGFR antibodies. For example, anti-EGFR antibodies can be produced by immunizing animals. A variety of methods have been described for preparing antigen for immunization and for generating monoclonal antibodies from immunized animals (see e.g., Kohler et al., Nature 256:495-497, 1975; Kohler and Milstein, Eur. J. Immunol. 6:511-519, 1976; Milstein et al., Nature 266:550-552,1977; Koprowski et al., U.S. Pat. No. 4,172,124; Harlow and Lane, 1988, Antibodies: A Laboratory Manual, (Cold Spring Harbor Laboratory Press: Cold Spring Harbor, N.Y.); Current Protocols In Molecular Biology, Vol. 2 (Supplement 27, Summer '1994), Ausubel et al., Eds. (John Wiley & Sons: New York, N.Y.), Chapter 11 (1991)). Generally, a hybridoma can be produced by fusing a suitable immortal cell line (e.g., a myeloma cell line) with antibody producing cells. The antibody producing cells, preferably those of the spleen or lymph nodes, are obtained from immunized animals. The fused cells (hybridomas) can be isolated using selective culture conditions and cloned by limiting dilution. Cells that produce antibodies with the desired specificity can be selected by a suitable assay (e.g., ELISA) and are within the scope of the present invention.

Other suitable methods of producing or isolating anti-EGFR antibodies include, for example, methods that rely upon immunization of transgenic animals (e.g., mice) capable of producing a full repertoire of human antibodies (see e.g., Jakobovits et al., Proc. Natl. Acad. Sci. USA 90: 2551-2555, 1993; Jakobovits et al., Nature 362: 255-258, 1993; Lonberg et al., U.S. Pat. No. 5,545,806; and Surani et al., U.S. Pat. No. 5,545,807).

Single chain antibodies, and chimeric, humanized or CDR-grafted antibodies, as well as chimeric or CDR-grafted single chain antibodies, comprising portions derived from different species, are also encompassed by the present invention and the term “antibody.” The various portions of these antibodies can be joined together chemically by conventional techniques, or can be prepared as contiguous polypeptides using genetic engineering techniques. For example, nucleic acids encoding a chimeric or humanized chain can be expressed to produce a contiguous polypeptide. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No.0,120,694 B1; Neuberger et al., WO 86/01533; Neuberger et al., European Patent No. 0,194,276 B1; Winter, U.S. Pat. No. 5,225,539; and Winter, European Patent No. 0,239,400 B1. See also, Newman et al. (BioTechnology 10:1455-1460, 1992) regarding CDR-graft antibodies, and Ladner et al. (U.S. Pat. No. 4,946,778) and Bird et al. (Science 242: 423-426, 1988) regarding single chain antibodies.

In addition, antigen binding portions of antibodies, including fragments of chimeric, humanized, CDR-grafted or single chain antibodies, can also be produced and are within the scope of the present invention. Antigen binding portions of the antibodies retain at least one binding function of the full-length antibody from which they are derived. Preferred antigen binding portions retain an antigen binding function of a corresponding full-length antibody (e.g., specificity for an EGFR). Functional fragments can retain the ability of the full-length antibody to inhibit one or more functions characteristic of an EGFR, such as kinase activity. As with the polypeptide-based targeting agents described above, the antibody-based targeting agents can modulate a constitutive activity of an EGFR (e.g., constitutively active phosphorylation of a ligand-independent receptor) or activation in response to ligand binding. For example, a targeting agent can modulate phosphorylation of an EGFR in response to binding of a ligand to the receptor. As with the polypeptide-based targeting agents, an antibody-based targeting agent may bind Domain II or Domain IV without inhibiting binding of a ligand to the antibody-bound receptor.

Antibodies useful in the present methods can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH₁ domain and hinge region of the heavy chain.

The invention provides chimeric antibodies that can be prepared as a contiguous polypeptide using genetic engineering techniques (e.g., DNA encoding the protein portions of the chimeric antibody can be expressed to produce a contiguous polypeptide chain). One example of a chimeric antibody provided herein is an antibody containing one or more antibody chains comprising a CDR (e.g., one or more CDRs of an antibody described herein) and a framework region derived from a light and/or heavy chain of a second antibody (e.g., of human origin; e.g., CDR-grafted antibodies with or without framework changes). Chimeric or CDR-grafted single chain antibodies also include humanized immunoglobulin. See, e.g., Cabilly et al., U.S. Pat. No. 4,816,567; Cabilly et al., European Patent No. 0,125,023 B1; Queen et al., European Patent No. 0,451,216 B1; Boss et al., U.S. Pat. No. 4,816,397; Boss et al., European Patent No. 0,120,694 B1; Neuberger et al., WO 86/01533; Neuberger et al., European Patent No. 0,194,276 B1; Winter, U.S. Pat. No. 5,225,539; Winter, European Patent No. 0,239,400 B1; Padlan et al., European Patent Application No. 0,519,596 A1. See also, Ladner et al., U.S. Pat. No. 4,946,778; Huston, U.S. Pat. No. 5,476,786; and Bird et al., Science 242:423-426, 1988), regarding single chain antibodies.

Chimeric antibodies can be produced using synthetic and/or recombinant nucleic acids to prepare genes (e.g., cDNA) encoding the desired chimeric chain. For example, nucleic acid (e.g., DNA) sequences coding for variable regions can be constructed using PCR mutagenesis methods to alter DNA sequences encoding an antibody chain, e.g., using methods employed to generate humanized antibodies (see e.g., Kanunan et al., Nucl. Acids Res. 17:5404, 1989; Sato et al., Cancer Research 53:851-856, 1993; Daugherty et al., Nucleic Acids Res. 19(9):2471-2476,1991; and Lewis and Crowe, Gene 101:297-302, 1991). Using these or other suitable methods, variants can also be readily produced. In one embodiment, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213).

The antibody or an antigen-binding portion thereof can include, for example, a sequence at least 80% identical (e.g., 85%, 90%, 95%, 98% or 100%) to one of the following sequences: SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36, SEQ ID NO:37; SEQ ID NO:38; or SEQ ID NO:39.

In various embodiments, the antibody or antigen-binding portion thereof includes a complementarity determining region (CDR) that is at least 40% identical (e.g., 40%, 60%, 80%, 85%, 90%, 95%, 98% or 100%) to a CDR of any one of SEQ ID NOs. 18-39.

The antibodies and antigen binding portions thereof described herein may have additional conservative or non-essential amino acid substitutions (a “non-essential” amino acid residue is a residue that can be altered from the wild-type sequence of a polypeptide, such as a binding agent, e.g., an antibody, without substantially altering a biological activity, whereas an “essential” amino acid residue results in such a change).

Whether or not a particular substitution will be tolerated, i.e., will not adversely affect desired biological properties, such as binding activity, can be determined by methods known in the art (e.g., as described in Bowie et al. (Science, 247:1306-1310, 1990)).

As described herein, the antibodies of the present invention can modulate (e.g., inhibit or activate) an activity of an EGFR and/or inhibit a function associated with EGFR activity, such as cell proliferation. As discussed below and elsewhere herein, various methods can be used to assess modulation of EGFR activity and/or a function (e.g., a cellular function or disease process or progression) associated with the activity.

Binding Assays

There are numerous ways to determine whether a polypeptide-based or antibody-based targeting agent binds (e.g., specifically binds) an EGFR and subsequently affects the cell in which the receptor is expressed in a desirable way. For example, a mammalian EGFR or a portion thereof can be maintained under conditions suitable for binding (e.g., in vivo or in cell culture); the EGFR (or the portion thereof) can be contacted with a potential targeting agent; and binding between the EGFR (or the portion thereof) and the potential targeting agent can be detected and, optionally, measured. An EGFR can be a receptor naturally expressed in cells. Alternatively, one can configure the assay using cells that are stably or transiently transfected with a construct comprising a nucleic acid sequence that encodes an EGFR or a portion thereof. In either event, the cells can be maintained under conditions appropriate for receptor expression and the receptor can be any of those described herein (e.g., EGFR, HER2, HER3, or HER4, including truncated versions thereof).

The cells can be contacted with a targeting agent under conditions suitable for binding (e.g., in the presence of a suitable binding buffer or under physiological conditions), and binding can be detected by standard techniques. The extent of binding can be determined relative to a suitable control. For example, receptor binding and/or activation in the presence of a potential targeting agent can be compared with a background level of binding and/or activation determined in the absence of a targeting agent. The comparison may also be made relative to the level of binding observed with a second, non-EGFR-specific polypeptide or antibody. In other embodiments, the assay can be configured to assess binding and/or activation relative to cells that do not express the EGFR (e.g., untransfected cells that do not naturally express an EGFR). A cellular fraction, such as a membrane fraction, containing an EGFR can be used in lieu of whole cells.

As noted above, the polypeptide or antibody targeting agent can be labeled with a suitable label (e.g., a fluorescent label, an isotope, an enzyme that catalyzes a reaction producing a detectable product, or another tag such as a FLAG tag (e.g., a polypeptide specifically bound by an antibody). Binding can be determined and assessed by detecting the label. The assessment can include determining the affinity with which the polypeptide- or antibody-based targeting agent binds the receptor. One can also determine the location of bound receptors. In other embodiments, a bound targeting agent can be detected by a labeled antibody. Binding specificity can be assessed by competition or displacement. For example, an unlabeled antibody or a ligand can be applied as a competitor.

Binding inhibition assays can also be used to identify binding agents that bind an EGFR (a receptor “of interest”) and inhibit not the receptor's natural ligand but another binding partner (e.g., a receptor that forms a homodimer or heterodimer with the receptor of interest). The receptor of interest may also be referred to as the first receptor and the receptor with which it dimerizes may be referred to as the second receptor. In specific embodiments, a first mammalian EGFR or a portion thereof can be maintained under conditions suitable for binding (e.g., in vivo or in cell culture); the first EGFR (or the portion thereof) can be contacted with a potential targeting agent; and binding between the first EGFR (or the portion thereof) and a second EGFR can be detected and, optionally, measured. The cells, receptors, reagents (e.g., buffers), and types of targeting agents used in this assay can be any of those described herein.

Other methods of identifying or assessing a targeting agent are available. These methods include other binding assays and methods that monitor one or more of the events that are triggered by EGFR activation (e.g., phosphorylation, cellular proliferation, or transformation). The cells may be cancer cells, and the cells or membrane fractions used in the assays may overexpress an EGFR or express a truncated variant associated with cancer. Inhibitory targeting agents can be identified as the agents that inhibit one or more of the downstream processes triggered by ligand binding. Inhibitory targeting agents may reduce the rate of cell division, the likelihood of transformation, cell motility, or another aspect of tumorigenicity.

Competition between polypeptide-based or antibody-based targeting agents can also be assessed, and the assays described here and known in the art can be used to assess diagnostic or therapeutic utility.

Diagnostic and Therapeutic Applications

The polypeptide- and antibody-based targeting agents described herein are useful in a variety of applications. For example, they can be used in screening assays, as diagnostic agents, and in therapeutic regimes. The targeting agents can consist of only a polypeptide that exhibits some extent of identity to the second or fourth domains of an EGFR or an anti-EGFR antibody that specifically binds those domains. Alternatively, the agents can include one or more of the heterologous entities described above (e.g., a fluorescent label, chemiluminescent label, isotope label, epitope or enzyme label or an agent that increases the polypeptide's circulating half-life).

Overexpression of EGFRs and EGFR variants are associated with malignant transformation and tumor resistance to chemotherapy and radiotherapy. The polypeptide- and antibody-based targeting agents described herein that block and/or inhibit the activity of an EGFR (e.g., an EGFR variant associated with cancer) can be used to reduce the likelihood of cell transformation; to increase the sensitivity of an EGFR-associated tumor to chemotherapy and/or radiotherapy; and/or to diagnose cancer, or a heightened risk thereof, in a patient. Accordingly, the present invention provides methods of inhibiting EGFR activity of a cell that expresses an EGFR or portion thereof. The methods can be carried out by contacting the cell with an effective amount of an inhibitory polypeptide- or antibody-based targeting agent that specifically binds the EGFR within Domain II or Domain IV. The cell can be a cell within the subject (e.g., a tumor cell), and the targeting agent can be administered to the subject in vivo. The targeting agents can be used prophylactically to treat a subject who is considered to be at risk for developing a cancer. We may refer to either a subject or patient. While the methods of the invention can be applied to any animal subject or patient, we expect application to be to human subjects or patients in many instances.

The polypeptide- or antibody-based targeting agents can be administered in combination with another therapeutic agent (or agents). For example, the inhibitory targeting agents described herein can be administered with another anti-cancer agent. Nonlimiting examples of anti-cancer agents include antitubulin and antimicrotubule agents, topoisomerase inhibitors, antimetabolites, mitotic inhibitors, alkylating agents, intercalating agents, agents that interfere with a signal transduction pathway, agents that promote apoptosis (including cell death genes), radioactive compounds, and antibodies against other tumor-associated antigens (including naked antibodies, immunotoxins and radioconjugates). The antitubulin and antimicrotubule agents include, for example, paclitaxel, vincristine, vinblastine, vindesine, vinorelbin, and taxotere; topoisomerase I inhibitors include, for example, topotecan, camptothecin, doxorubicin, etoposide, mitoxantrone, daunorubicin, idarubicin, teniposide, amsacrine, epirubicin, merbarone, and piroxantrone hydrochloride; antimetabolites include, for example, 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate (PALA), pentostatin, 5-azacitidine, 5-Aza 2′-deoxycytidine, ara-A, cladribine, 5-fluorouridine, FUDR, tiazofurin, and N-[5-[N-(3,4-dihydro-2-methyl-4-oxoquinazolin-6-ylmethyl)-N-methylamino]-2-thenoyl]-L-glutamic acid; alkylating agents include, for example, cisplatin, carboplatin, mitomycin C, melphalan, thiotepa, busulfan, chlorambucil, plicamycin, dacarbazine, ifosfamide phosphate, cyclophosphamide, nitrogen mustard, uracil mustard, pipobroman, and 4-ipomeanol; agents acting via other mechanisms include dihydrolenperone, spiromustine, and desipeptide; biological response modifiers include interferon; apoptotic agents include actinomycin D; and anti-hormones include anti-estrogens such as tamoxifen and antiandrogens such as 4′-cyano-3-(4-fluorophenylsulphonyl)-2-hydroxy-2-methyl-3′-(trifluoromethyl)propionanilide.

The polypeptide- and antibody-based targeting agents also have value in diagnostic applications. These agents can be used to monitor the growth and/or the metastasis of a tumor in vivo, and they may be used as a diagnostic indicator of the stage to which a disease has progressed (e.g., as an indicator of the grade of cancer). Where the agent is an antibody, it may be preferable to use human or humanized antibodies to carry out an in vivo diagnostic test on a human patient.

For diagnostic purposes, the targeting agents can be labeled or unlabeled. The diagnostic assays may entail detecting the formation of a complex that includes the targeting agent and the EGFR. As noted, the targeting agents can be labeled, and the label can be a radionuclide, a fluorescent molecule, an enzyme, an enzyme substrate, an enzyme cofactor, an enzyme inhibitor, or a ligand (e.g., biotin, haptens, and the like). Numerous immunoassays are known in the art and can be adopted for use with the targeting agents described herein (see, for example, U.S. Pat. Nos. 3,817,827; 3,850,752; 3,901,654 and 4,098,876). Unlabeled targeting agents can also be used in combination with other (i.e., one or more) suitable reagents, such as a labeled antibody (e.g., a second antibody) reactive with the first antibody (e.g., anti-idiotype antibodies or other antibodies that are specific for the unlabeled immunoglobulin) or other suitable reagents (e.g., labeled protein A).

The invention also encompasses kits that contain one or more reagents useful in conducting the diagnostic or therapeutic methods of the present invention. For example, the kits can contain one or more reagents for detecting the presence of an overexpressed or truncated EGFR in a biological sample. Such kits can include a polypeptide- or antibody-based targeting agent and one or more ancillary reagents suitable for detecting the presence of a complex between the targeting agent and the EGFR. The agents can be provided in lyophilized form, either alone or in combination with additional antibodies specific for other epitopes. The agents, whether labeled or unlabeled, can be included in the kits with adjunct ingredients (e.g., buffers, such as Tris, phosphate and carbonate, stabilizers, excipients, biocides and/or inert proteins, e.g., bovine serum albumin). For example, the agents can be provided as a lyophilized mixture with the adjunct ingredients, or the adjunct ingredients can be separately provided for combination by the user. Where a secondary antibody is employed, the secondary antibody can also be provided in the kit (for instance in a separate vial or container). The secondary antibody, if present, is typically labeled, and can be formulated in an analogous manner with the formulations described above.

The polypeptide- and antibody-based targeting agents of the present invention can be used not only to detect, but also to quantitate expression of an EGFR by a cell. The methods can be carried out by providing a cell or a fraction thereof (e.g., a membrane fraction) and contacting the cell or cell fraction with an agent that binds to the EGFR (e.g., Domain II or Domain IV of an EGFR). The extent of antibody binding can be monitored and/or quantitated. Detection of the agent (e.g., an anti-EGFR antibody) indicates formation of a complex between the agent and an EGFR and thereby indicates the presence of an EGFR. Binding of the antibody to the cell can be determined as described above. The method can be used to detect expression of EGFR in cells from an individual (e.g., in a tumor biopsy sample). A quantitative expression of EGFR on the surface of tumor cells can be evaluated, for instance, by flow cytometry, and the staining intensity can be correlated with disease progression or risk.

The polypeptide- and antibody-based targeting agents described herein can be used to inhibit tumor formation, growth, or metastases and/or to enhance an EGFR-associated tumor's sensitivity to chemotherapy or radiotherapy. The inhibition may be, but is not necessarily, complete inhibition. The targeting agents described herein may inhibit the receptor to any extent that produces a physiological or clinical benefit upon a subject to whom they are administered. Where the targeting agent is an inhibitory agent, it can be used to inhibit any signal mediated by an EGFR; where the targeting agent is a stimulatory agent, it can be used to stimulate any signal mediated by an EGFR.

Diseases that can be treated include EGFR-associated cancers. Moreover, these cancers include, but are not limited to, solid tumors, soft tissue tumors, and metastatic lesions. Examples of solid tumors include malignancies, e.g., sarcomas, adenocarcinomas, and carcinomas, of the various organ systems, such as those affecting the lung, breast, lymphoid, gastrointestinal (e.g., colon), and genitourinary tracts (e.g., renal and urothelial cells), the liver, pharynx, prostate, or ovary, cholangiocarcinomas, as well as adenocarcinomas, which include malignancies such as most colon cancers, rectal cancer, renal-cell carcinoma, liver cancer, non-small cell carcinoma of the lung, cancer of the small intestine, neuroectodermal tumors, and the like.

Metastatic lesions of the aforementioned cancers can also be treated or prevented (which may include a reduction in the size, growth rate, or spread of cancerous cells) using the methods and compositions of the invention. In various embodiments, the inhibitory targeting agents described herein are used for treatment of glioblastoma, squamous cell cancer, non-small cell lung cancer, breast cancer, colon cancer, or prostate cancer.

The methods of the present invention can be used to treat malignancies of various organ systems, such as those affecting the lung, breast, lymphoid system, gastrointestinal (e.g., colon), and genitourinary tracts, the prostate, ovary, and pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term carcinomas also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is recognized by those skilled in the art and refers to malignant tumors of mesenchymal derivation.

The methods of the invention can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, Crit Rev. in Oncol./Hemotol. 11:267-297, 1991). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T cell lymphomas, adult T cell leukemia/lymphoma (ATL), cutaneous T cell lymphoma (CTCL), large granular lymphocytic leukemia (LGF) and Hodgkin's disease.

Modes of Administration

One or more of the polypeptide- or antibody-based targeting agents described herein can be administered to a subject or patient by an appropriate route, either alone or in combination with another drug. When administered in combination, the therapeutic agents of the present invention can be administered before, simultaneously with, or after administering another drug by the same or a different route of administration. As noted above, the agents of the present invention can be used in combination with a (or another) monoclonal or polyclonal antibody or with a chemotherapeutic agent or treatment.

An effective amount of a targeting agent is administered. An effective amount is an amount sufficient to achieve the desired therapeutic effect, under the conditions of administration. In the present case, an effective amount can be an amount sufficient to inhibit one or more of the functions of an EGFR that are associated with an undesirable condition (e.g., cancer or risk of cancer) and thereby, inhibit that condition (i.e., cancer or risk of cancer).

A variety of routes of administration are possible. These routes include oral and parenteral routes of administration. The parenteral route can be, for example, an intravenous, intraarterial, intramuscular, or. subcutaneous injection. The targeting agents can also be administered topically or by inhalation (e.g., intrabronchial, intranasal or oral inhalation or by intranasal drops), depending on the disease or condition to be treated. Where sustained delivery is desired, the targeting agents can be delivered by rechargeable or biodegradable devices (e.g., polymeric devices) that release the agents over time.

Formulation of the targeting agents will vary according to the route of administration selected. For example, an agent is likely to be formulated as a capsule or tablet for oral delivery; as a gel for topical delivery; or as solution or suspension for intravenous or intramuscular delivery. When formulated as a pharmaceutical formulation, the targeting agents described herein can be prepared in physiologically acceptable diluents. These formulations can include more than one (e.g., two, three, four, or more) types of targeting agents. For solutions or emulsions, suitable diluents include aqueous or alcoholic/aqueous solutions, saline, and buffered media (e.g., phosphate-buffered saline). Parenteral vehicles can include sodium chloride solution, Ringer's dextrose, dextrose and sodium chloride, lactated Ringer's or fixed oils. A variety of appropriate aqueous diluents are known in the art. These include water, buffered water, buffered saline, polyols (e.g., glycerol, propylene glycol, liquid polyethylene glycol), dextrose-containing solutions and glycine. The formulations can include various additives, preservatives, or fluid, nutrient or electrolyte replenishers (see, generally, Remington's Pharmaceutical Science, 16th Edition, Mack, Ed. 1980). The compositions can optionally contain pharmaceutically acceptable auxiliary substances as required to approximate physiological conditions. These substances include pH adjusting and buffering agents and toxicity adjusting agents (e.g., sodium acetate, sodium chloride, potassium chloride, calcium chloride and sodium lactate).

Any of the targeting agents can be lyophilized for storage and reconstituted in a suitable carrier prior to use. The optimum concentration of the active ingredient(s) in the chosen medium can be determined empirically, according to procedures well known to those of ordinary skill in the art, and will depend on the ultimate pharmaceutical formulation desired. For example, for inhalation, the compound can be solubilized and loaded into a suitable dispenser for administration (e.g., an atomizer, nebulizer or pressurized aerosol dispenser).

A number of embodiments of the invention have been described. Nevertheless, it will be understood that various modifications may be made without departing from the spirit and scope of the invention.

### EXAMPLES

The invention is further described in the following examples, which do not limit the scope of the invention described in the claims.

### Example 1

### Selection and Characterization of EGFR Polypeptide Fragments

We selected amino acid sequences corresponding to fragments of Domain II and Domain IV of EGFR. The sequence of each polypeptide is shown in the first column of Table 1. The polypeptides were chosen based on their positions on the crystal structures of monomeric and dimeric EGFR (Burgess et al., Mol. Cell 12:541-552, 2003). Although Domain IV of EGFR is not resolved in the dimer crystal structure, it is believed that portions of Domain IV come into contact upon dimerization. By binding to polypeptides in Domains II and IV, we believe it is possible to sterically inhibit EGFR dimerization and subsequent phosphorylation and signaling. We generated biotinylated cyclic polypeptides that included each sequence and tested them in a model HMEC cell system. The sequences of the biotinylated cyclic polypeptides are shown in the third column of Table 1. Of the cyclic polypeptides containing a sequence derived from Domain II of EGFR, only one failed to inhibit EGFR phosphorylation. We also tested rabbit polyclonal antisera generated against two of the polypeptides and found that the antisera inhibited EGFR phosphorylation in 184A1 human mammary epithelial cells (HMECs) (Band and Sager, PNAS 86:1249-1253, 1989; Hendriks et al., Cancer Res. 63, 1130-1137, 2003). For the LYNPTTYQMD polypeptide, the different results obtained from polypeptide and polyclonal antisera inhibition could be due to differences in the quantitative details of the assays, or the polypeptide could have a lower affinity for the receptor than the polyclonal antisera does.

TABLE 1
EGFR Peptide Sequences and Peptide and Antisera Activity

| | Domain | | | Polyclonal |
|---|---|---|---|---|
| Peptide | (residues | | Peptide | antisera |
| sequence | of EGFR) | Cyclic peptide sequence | Inhibition | inhibition |
| LYNPTTYQMD | II | Biotin-GCGLYNPTTYQMDGCG- | No | Yes |
| (SEQ ID NO:1) | (245-254) | CONH₂(SEQ ID NO:7) | | |
| CRNVSRGREC | IV | Biotin-GCRNVSRGRECG- | Yes | |
| (SEQ ID NO:2) | (502-511) | CONH₂(SEQ ID NO:8) | | |
| CAHYIDGPHC | IV | Biotin-GCAHYIDGPHCG- | Yes | |
| (SEQ ID NO:3) | (558-567) | CONH₂(SEQ ID NO:9) | | |
| MGENNT | IV | Biotin-Aha-CMGENNTG- | Yes | Yes |
| (SEQ ID NO:4) | (576-581) | CONH₂(SEQ ID NO:10) | | |
| LVWKYADAG | IV | Biotin-GCLVWKYADAGCG- | Yes | |
| (SEQ ID NO:5) | (582-590) | CONH₂(SEQ ID NO:11) | | |

Aha = 6-aminohexanoic acid

We also attempted to synthesize a peptide that included the following sequence from Domain II of HER2: TYNTDTFESM (SEQ ID NO: 12). The sequence of the polypeptide we attempted to synthesize was: Biotin-GCGTYNTDTFESMGCG-CONH₂ (SEQ ID NO: 13). Synthesis of this peptide was not successful. We synthesized two polypeptides of HER2 containing the following sequences from domain IV: KPDLSYM (SEQ ID NO: 14) and MPIWKFPDEE (SEQ ID NO: 15). The sequences of these peptides were Biotin-Aha-CKPDLSYMCG-CONH₂ (SEQ ID NO: 16) and Biotin-GC MPIWKFPDEECG-CONH₂ (SEQ ID NO: 17).

### Example 2

### Screen for Antibodies to EGFR Peptide Fragments

We screened a yeast display library encoding human immunoglobulin single chain variable fragments (scFvs) and variable heavy chain domains (heavy chains, VHs) for those that bind to the polypeptides described in Example 1. The sequences of the heavy chains and scFvs that we isolated are depicted in Table 2, below.

The naïve human single-chain Fv library was expressed in yeast using a yeast surface display technique (Boder and Wittrup, Nat Biotechnol. 15(6):553-7, 1997). This library was prepared as described in Feldhaus et al. (Nature Biotech. 21:163-170, 2003). Briefly, human antibody variable genes were cloned by PCR from commercially available spleen and lymph node poly(A) mRNA pooled from 58 adults. Primers to IgG, IgM, κ and λ were used for first-strand cDNA synthesis. Separate VH and VL libraries were constructed, then assembled together in single-chain Fv (scFv) format by overlap extension PCR. The scFv library was then subcloned for expression as an Aga2p fusion on the yeast surface. A library of approximately 10⁹ scFv fragments was expressed in yeast. This scFv library also contained VH domains resulting from gene truncation or frameshifts.

The library was screened for binding to the polypeptides in Table I as described in Boder and Wittrup (Biotechnol Prog. 14(1):55-62, 1998; Methods Enzymol. 328:430-444, 2000). Briefly, the library was screened against the polypeptides using one round of magnetic cell sorting (MACS) and three rounds of fluorescence activated cell sorting (FACS) to obtain the clones listed in Table 2. VH clones were isolated from the library to bind specifically to each of the peptides except CAHYIDGPHC (SEQ ID NO:3), for which full scFvs were isolated. The affinities of these isolated clones were estimated to be 1-10 μM based on partial yeast cell surface labeling at these concentrations. The isolated VH clones were then paired with a variety of light chains from the original library using PCR and DNA homologous recombination in yeast. These new pairings were screened for retention of binding to the polypeptides using 3-5 rounds of FACS.

TABLE 2

Amino Acid Sequences of Variable Heavy
Chains and scFvs Isolated from Naïve
Library Screen

| | |
|---|---|
| Peptide | VH/scFv Sequence |

| | |
|---|---|
| LYNPTTYQMD | QVQLVQSEGGVVQPGRSLRLSCAASGFTFSSYAMHWV |
| (SEQ ID NO:1) | RQAPGKGLEWVAVISYDGSNKYYADSVKGRFTISRDN |
| | SKNTLYLQMNSLRAEDTAVYYCARDEGYYGSGCIDYW |
| | GQGTLVTVSSGILGSGGGSGGGGSGGGGLQEF |
| | (SEQ ID NO:18) |
| LYNPTTYQMD | QVQLVQSEGGVVQPGRSLRLSCAAPGFTFSSYAMHWV |
| (SEQ ID NO:1) | RQAPGKGLEWVAVISYDGSNKYYADSVKGRFTISRDN |
| | SKNTLYLQMNSLRAEDTAVYYCARDEGYYGSGCIDYW |
| | GQGTLVTVSSGILGSGGGGSGGGGSGGGGLQEF |
| | (SEQ ID NO:19) |
| CRNVSRGREC | QVQLQQSGPGLVKPSQTLSLTCDISGDSVSSDSAAWN |
| (SEQ ID NO:2) | WIRLSPSRGLEWLGRTYYWSKWYTDYAVSVKSRITIN |
| | PDTSKNQFSLQLNSVTPEDTAVYFCARERYCSSTSCY |
| | LDAFDIWGQGTMVTVSGILGSGGGGSGGGGSGGGGLQ |
| | EF |
| | (SEQ ID NO:20) |
| CRNVSRGREC | QVQLQQWGPGLLKPSETLSLTCGVSGGSLSGYYWSWI |
| (SEQ ID NO:2) | RQSPGKELEWIGEINQGGSTNYNPSLRSRVTISVDTS |
| | KKQLSLKVNSVTASDTAVYYCARETFRGSNCFDSWGQ |
| | GTLVTVSSGILGSGGGIGGGGSGGGGLQEF |
| | (SEQ ID NO:21) |
| CAHYIDGPHC | QVQLQQSGPGLVKPSQTLSLTCAISGDSVSSNTAAWN |
| (SEQ ID NO:3) | WIRQSPSRGLEWLGRTYYRSKWHNDYAASVKSRITIN |
| | PDTSKNQFSLQLNSVTPEDTAVYYCARGRGVWDFDIW |
| | GRGTLVTVSSGILGSGGGGSGGGGSGGGGSQPVLTQS |
| | PSVSVAAGETARVTCGGNSFRRRSVCWYQQRPGQAPV |
| | LVVYDDSDRPSGIPARFSGSNSGNVATLTISSVEAGD |
| | EADYYCQCGIVTMIMWFSAEGQADRPIRNSRQKLISE |
| | EDL |
| | (SEQ ID NO:22) |
| CAHYIDGPHC | QVQLQQSGPGLVKPSQTLSLTCAISGDSVSSNRAAWN |
| (SEQ ID NO:3) | WIRQSPSRGLEWLGRTYYRSKWYYDYTLSVQGRITIS |
| | PDTSRNQVFLQLNYVTPEDTAVYYCARVPQDSSGSRH |
| | DAFDFWGQGTMVTVSSGILGSGGGGSGGGGSGGGGSQ |
| | PVLTQSPSASGTPGQRVTLSCAGSSSDIGSNPVNWYQ |
| | QLPGTAPKLLMYSNDQRPSGVSDRFSGSKSGTSASLA |
| | VSGLQSEDEADYYCAAWDDSLNGWVFGGGTKLTVLSG |
| | ILEQKLISEEDL |
| | (SEQ ID NO:23) |
| CAHYIDGPHC | QVQLVQSGAEVKKPGASVKISCKASGYTFTRYYMHWV |
| (SEQ ID NO:3) | RQAPGQGLEWMGIINPSGGTTSYAEKFQGRVTMTRGT |
| | STRTVYMELSSLGSEDTAVYFCVRDLVDVDDYSGLHA |
| | FDIWGQGTMVTVSSGILGSGGGGSGGGGSGGGGSDIV |
| | MTQTPATLSVSPGGGATLSCRGQSEYRQQRSLVPAET |
| | WPGSQAPHVWCIHEGHWYPSQVQWQCVWDRFHPHHQQ |
| | TGS |
| | (SEQ ID NO:24) |
| CAHYIDGPHC | QVQLQQSGPGLVKPSQTLSLTCAISGDTVSSNSAAWH |
| (SEQ ID NO:3) | WIRQSPSRGLEWLGRTYYRSKWYNEYAVSVKSRITIN |
| | PDTSKNQFSLQLDSVTPEDTAVYYCVRDFYVGFAYWG |
| | QGTLVTVSSGILGSGGGGSGGGGSGGGGSEIVLTQSP |
| | GTLSLSPGERATLSCRASQSVSSSYLAWYQQKPGQAP |
| | RLLIYGASSRATGIPDRFSGSGSGTDFTLTISRLEPE |
| | DFAVYYCQQYGSSFTFGPGTKVDIKSGILEQKLISEE |
| | DL |
| | (SEQ ID NO:25) |
| CAHYIDGPHC | EVQLVESGGGLVQPGGSLRLSCAASGFTFSNSAMSWV |
| (SEQ ID NO:3) | RQAPGKGLEWVSVIHSGGSTTYYADSVKGRFTISRDN |
| | SKNTLYLQMSSLRAEDTAVYYCAKHTSTWWGGEDHYG |
| | MDVWGQGTTVTVSSGILGSGGGGSGGGGSGGGGSQSV |
| | LTQPASVSGSPGQSVTISCTGTSSDVGSYMFVSWYQQ |
| | HPGKAPKLMISDVSNRPSGVPDRFSGSKSGNTASLTI |
| | SGLRAEDEADYYCSSYTSTNTRVFGGGTKLTVLSGIL |
| | EQKLISEEDL |
| | (SEQ ID NO:26) |
| CAHYIDGPHC | QITLKESGPTLVKPTQTLTLTCTFSGFSLSTNGVGVG |
| (SEQ ID NO:3) | WIRQPPGKALEWLALIYWNDDKRYSPSLKRRLTITKD |
| | TSKNQVVLTMTNMDPVDTATYYCAHRLLDSGYILPPT |
| | FDYWGQGTLVTVSSGILGSGGGGSGGGGSGGGGSEIV |
| | LTQSPGTLSLSPGERATLSCRASQSVNSGYLAWYQQK |
| | PGQAPRLLISAASSRATGIPDRFSGSGSGTDFTLTIS |
| | RLEPEDFAVYYCQRYDTSRGFTFGPGTKVDIKSGF |
| | (SEQ ID NO:27) |
| CAHYIDGPHC | QVQLQQSGPGLVKPSQTLSLTCAISGDSVSSNSAAWN |
| (SEQ ID NO:3) | WIRQSPSRGLEWLGRTYYRSKWYNEYAVSVKSRITIN |
| | PDTSKNHFSLQLNSVTPEDTAVYYCAREIVGTPFDYW |
| | GQGTLVTVSSGILGSGGGGSGGGGSGGGGSDIRVTQS |
| | PSSLSASVGDRVTITCRTSQSASKYVNWYQQKPGKVP |
| | KLLIYAASSLQSGVPSRFSGSGSGTHFTLSISNLQPG |
| | DMGTYYCQQGYGTPLTFGGGTKLEIKSGF |
| | (SEQ ID NO:28) |
| CAHYIDGPHC | QVQLQQSGPGLVKPSQTLSLTCAISGDSVSSNSAAWN |
| (SEQ ID NO:3) | WIRQSPSRGLEWLGRTAYRSKWNSDYAASVRSRITIN |
| | PDTSKNQFSLQLNSVTPDDTAMYYCARSRSSSPIDYW |
| | GQGTLVTVSSGILGSGGGGSGGGGSGGGGSEIVLTQS |
| | PGTLSLSPGERVTLSCRASQRVSSSYVAWYQQKPGQA |
| | PRLLIYGASSRGHWHPRQVQWQWVWDRLHSHHQQTGA |
| | (SEQ ID NO:29) |
| CAHYIDGPHC | QVQLKQSGPGLVNPSQTLSLTCAISGDSVSSDSAAWN |
| (SEQ ID NO:3) | WIRQSPSRGLEWLGRTYYRSKWYNEYTESVKSRIIIN |
| | PDTSKNQFSLQLNSVTPEDTALYYCARFLRGTFDIWG |
| | QGSMVTVSSASTKGPSGILGSGGGGSGGGGSGGGGSN |
| | FMLTQPHSVSESPGKTVTISCTGSSGSIATNYVQWYQ |
| | QRPGSAPTTVIYENNHRPSGVPDRFSGSIDSSSNSAS |
| | LTISGLKTEDEAGYYCQSSDNDFHAVFGGGTQLTVLS |
| | GILEQKLISEEDL |
| | (SEQ ID NO:30) |
| CAHYIDGPHC | QVQLVESEGGVVQPGRSLRLSCAASGFSFSRFAMHWV |
| (SEQ ID NO:3) | RQAPGKGLEWVAVISYDGSNKFYADSVKGRFTISRDN |
| | SKNTLYLQMDSLRAEDTAVYYCARHYDSSGRDHWGQG |
| | TLVTVSSGILGSGGGGSGGGGSGGGGSEIVMTQSPGT |
| | LSSSPGERATLSCRASKDVSSQFLAWYQQKPGQAPRL |
| | LIYETSTRGHWHPRQVQWQWVWDRLHSHHQQTGA |
| | (SEQ ID NO:31) |
| CAHYIDGPHC | QVQLQQSGPGLVKPSQTLSLTCAISGDSVSSNSAAWN |
| (SEQ ID NO:3) | WIRQSPSRGLEWLGRTAYRSKWNSDYAASVRSRITIN |
| | PDTSKNQFSLQLNSVTPDDTAMYYCARSRSSSPIDYW |
| | GQGTLVTVSSGILGSGGGGSGGGGSGGGGSEIVLTQS |
| | PGTLSLSPGERVTLSCRASQRVSSSYVAWYQQKPGQA |
| | PRLLIYGASSRTLASQTGSVAVGLGQTSLSPSADWSL |
| | KISQCITVSSMIVHLERSARDQAGDQILNSRQSLFLK |
| | RLVIASAAASRSDNNSVDVQIDFVPLYFSSYKYIYFH |
| | SP |
| | (SEQ ID NO:32) |
| CAHYIDGPHC | EVQLVESGGGLVQPGGSLRLSCAASGFTFSNSAMSWV |
| (SEQ ID NO:3) | RQAPGKGLEWVSVIHSGGSTTYYADSVKGRFTISRDN |
| | SKNTLYLQMSSLRAEDTAVYYCAKHTSTWWGGEDHYG |
| | MDVWGQGTFVTVSSGILGSGGGGSGGGGSGGGGSQSV |
| | LTQPASVSGSPGQSVTISCTGTSSDVGSYMFVSWYQQ |
| | HPGKAPKLMISDVSNRPSRVPDRFSGSKSGNTASLTI |
| | SGLWAEDEADYYCSSYTSTNTRVFGEGPSSPSYPESR |
| | TKAYF |
| | (SEQ ID NO:33) |
| CAHYIDGPHC | EVQLVESGGGLVQPGRSLRLSCAASGFSFADYAMHWV |
| (SEQ ID NO:3) | RQVPGKGLEWVSSISWNSGIKGYADSVKGRFTISRDN |
| | AKNTVYLEVNNIRPEDTALYYCAKVRDPNIEAFDVWG |
| | QGTMVTVSSGILGSGGGGSGGGGSGGGGSEIVLTQSP |
| | GTLSLSPGERATLSCRASQSVSSSYLGWYQQKPGQAP |
| | RLLIYGASSRATGIPDRFSGSGSGTDFALTISRLEPE |
| | DFAVYYCQQHDRSSWTFGQGTKVEIKSGILEQKLISE |
| | EDL |
| | (SEQ ID NO:34) |
| CAHYIDGPHC | QVQLVESEGRLVKPGGSLRLSCVASGMTFNNAWMSWV |
| (SEQ ID NO:3) | RQAPGKGLEWVGRIRSESNGGTTDYAAPGKGRFTISR |
| | DDSKNTLYLQMNSLQIEDTAVYYCATDRGYSNSGNYY |
| | RHYWGQGTLVTVSSGILGSGGGGSGGGGSGGGGSDIR |
| | LTQSPSSLSASVGDRVTITCQASQDISNYLNWYQQKP |
| | GKAPKLLIYDASNLETGVPPRFSGSGSGTAFTFTISS |
| | LQPEDFATYYCQQYDNLPHTFGPGTKVDIKSGILEQK |
| | LISEEDL |
| | (SEQ ID NO:35) |
| MGENNT | QVQLQESGPGLVKPSETLSLTCTFSGGSIRSSSDYWG |
| (SEQ ID NO:4) | WVRQPPGKGLEWIGSISSSGSTYYNPSLKSRVTISVD |
| | TSRNQFSLRLSSVTAADTAIYYCARYNHYWYFDLWGR |
| | GTLVTVSSGILGSGGGSGGGGSGGGGSKRHSRSLQHS |
| | SQRLQETKSTSPAKPAKTLMMI |
| | (SEQ ID NO:36) |
| MGENNT | QVQLQESGPGLVKPSETLSLTCTVSGGSISNSDYYWG |
| (SEQ ID NO:4) | WIRQPPGKGLRWIGSINYYGTTYYNPSLKSRVAMSVD |
| | TSKNQFSLKLSSVTAADTAVYYCARRGANSWFFDLWG |
| | RGTLVTVSSGILGSGGGSGGGGSGGGGLQEF |
| | (SEQ ID NO:37) |
| LVWKYADAG | QITLKESGPTVVKPTQTLTLTCTFSGFSLSTSGVGVG |
| (SEQ ID NO:5) | WIRQPPGKALEWLALISWDDDKRYSPSLKSRLTITKD |
| | TSKNQVVLTMTNMDPVDTATYFCAHLNNFVDAIELRT |
| | GWCFDVWGRGTLVTVSGILGSGGGGSGGGGSGGGVLK |
| | LC |
| | (SEQ ID NO:38) |
| LVWKYADAG | QITLKESGPTVVKPTQTLTLTCTFSGFSLSTSGVGVG |
| (SEQ ID NO:5) | WIRQPPGKALEWLALISWDDDKRYSPSLKSRLTITKD |
| | TSKNQVVLTMTNMDPVDTATYFCAHLINFVDAIELRT |
| | GWCFDVWGRGTLVTVSGILGSGGGGSGGGGSGGGVLK |
| | LR |
| | (SEQ ID NO:39) |

### Example 3

### Characterization of Polyclonal Antisera Against EGFR Domain II and Domain IV Peptides

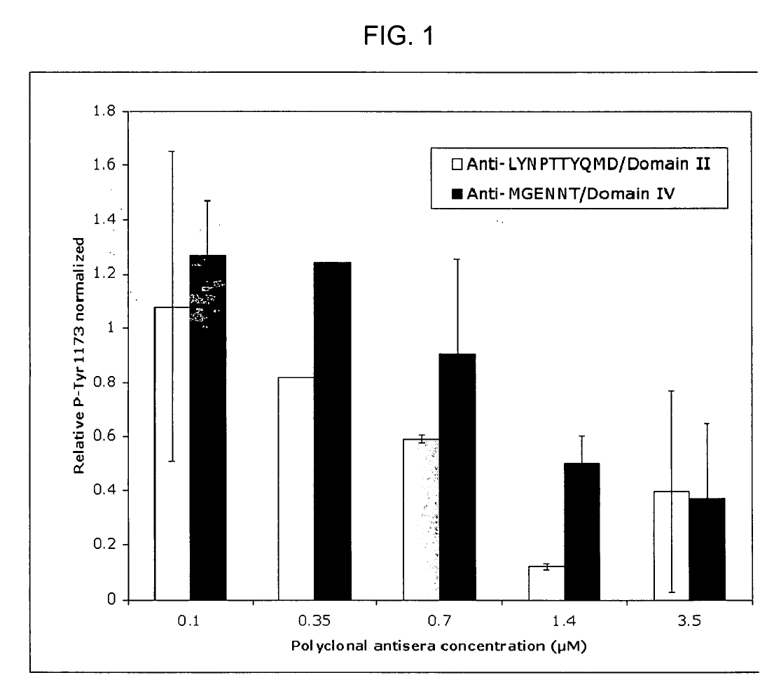

Anti-domain II and anti-domain IV rabbit polyclonal antisera were generated against polypeptides LYNPTTYQMD (SEQ ID NO: 1) and MGENNT (SEQ ID NO:4). Briefly, rabbits were injected with one of the peptides, and the resulting antisera were collected. The rabbit sera were purified by ammonium sulfate precipitation followed by desalting. These polyclonal antisera were then used to treat human mammary epithelial cells (HMECs) and to measure the antisera's effect on EGFR tyrosine 1173 phosphorylation by quantitative Western blot analysis. HMECs were grown to 80% confluence and incubated for 2 hours in serum-free media containing various concentrations of the rabbit polyclonal antisera. The cells were then washed and incubated with 25 nM EGF for 5 minutes. The cells were lysed, and the proteins in the lysate were run on a SDS-PAGE gel and transferred onto PDVF membranes. Blots were detected using an anti-EGFR phosphotyrosine 1 173 antibody and horseradish peroxidase, and band intensity was quantified using Kodak® ID 3.5 software. The results are shown in FIG. 1 as relative EGFR tyrosine 1173 phosphorylation in the presence of varying concentrations of polyclonal antisera. The tyrosine phosphorylation in the presence of antiserum has been normalized by total EGFR intensity and tyrosine phosphorylation in the absence of antiserum. These results show that antibodies that bind to polypeptides LYNPTTYQMD (SEQ ID NO: 1) and MGENNT (SEQ ID NO:4) can decrease the tyrosine phosphorylation of EGFR.

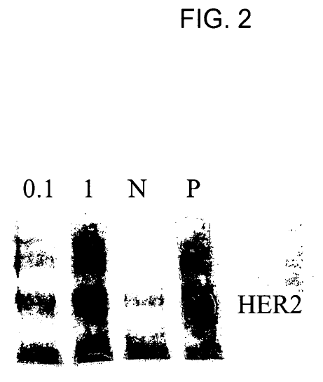

Next, we analyzed the amount of HER2 co-immunoprecipitated with EGFR in the presence and absence of rabbit polyclonal antiserum against peptide MGENNT. HMECs were treated as before with the antiserum. EGFR in each sample was immunoprecipitated from HMEC lysates using an anti-EGFR antibody obtained from Santa Cruz Biotechnology, Inc. Immunoprecipitates were resolved by SDS-PAGE and blotted with anti-HER2 antibody (Cell Signaling). The results are depicted in FIG. 2, where N is the non-EGF stimulated negative control, P is the cells with no rabbit antisera incubation, and 0.1 and 1 are the concentrations of rabbit polyclonal antisera in μM. In FIG. 2, less HER2 is co-immunoprecipitated with EGFR

What is claimed is:

1. A substantially pure polypeptide comprising 5-20 contiguous amino acid residues that are identical to an amino acid sequence present within the second domain or the fourth domain of a receptor in the epidermal growth factor receptor (EGFR) family, with the proviso that the polypeptide is not a naturally occurring receptor in the EGFR family or a polypeptide consisting of the sequence CAHYIDGPHC (SEQ ID NO:3), LVWKYADAG (SEQ ID NO:5), or CGADSYEMEEDGVRKC (SEQ ID NO:6).

2. The polypeptide of claim 1, wherein the polypeptide, upon contacting the receptor under physiological conditions, inhibits an activity of the receptor.

3. The polypeptide of claim 2, wherein the activity is tyrosine kinase activity.

4. The polypeptide of claim 2, wherein the activity is receptor dimerization.

5. The polypeptide of claim 1, wherein the polypeptide, upon contacting a biological cell that overexpresses the receptor, inhibits the rate at which the cell proliferates.

6. The polypeptide of claim 1, wherein the polypeptide, upon contacting a biological cell that expresses a truncated form of the receptor, inhibits the rate at which the cell proliferates.

7. The polypeptide of claim 5, wherein the cell is a cancer cell.

8. The polypeptide of claim 1, wherein the polypeptide, upon contacting the receptor under physiological conditions, does not prevent the receptor from binding its cognate ligand.

9. The polypeptide of claim 1, wherein the receptor is an EGFR, HER2, HER3, or HER4.

10. The polypeptide of claim 1, wherein the polypeptide is cyclic.

11. The polypeptide of claim 1, wherein the polypeptide consists of 5-20 contiguous amino acid residues.

12. The polypeptide of claim 1, wherein the polypeptide consists of 6-11 contiguous amino acid residues.

13. The polypeptide of claim 1, wherein the polypeptide further comprises a detectable moiety.

14. The polypeptide of claim 13, wherein the detectable moiety is biotin or an epitope tag.

15. The polypeptide of claim 1, comprising 5-20 contiguous amino acid residues that are identical to an amino acid sequence present within the fourth domain of a receptor in the EGFR family.

16. The polypeptide of claim 15, wherein the polypeptide comprises the sequence LYNPTTYQMD (SEQ ID NO:1); CRNVSRGREC (SEQ ID NO: 2); MGENNT (SEQ ID NO: 4); or a variant of any of SEQ ID NOs. 1, 2 or 4 in which one, two, or three amino acid residues are mutated.

17. The polypeptide of claim 1, comprising 5-20 contiguous amino acid residues that are identical to an amino acid sequence present within the second domain of a receptor in the EGFR family.

18. The polypeptide of claim 17, wherein the polypeptide comprises the sequence LYNPTTYQMD (SEQ ID NO: 1) or a variant thereof in which one, two, or three amino acid residues are mutated.

19. The polypeptide of claim 1, wherein the receptor is a human receptor.

20. A method of inhibiting an activity of a receptor in the EGFR family, the method comprising:
providing a cell that expresses the receptor and
contacting the cell, under physiological conditions, with an amount of the polypeptide of claim 1 that is sufficient to inhibit an activity of the receptor.

21. The method of claim 20, wherein the cell is a cell maintained in culture.

22. The method of claim 20, wherein the cell is a cell in a living subject.

23. A kit comprising the polypeptide of claim 1 and instructions for diagnostic or therapeutic use.

24. A pharmaceutically acceptable composition comprising the polypeptide of claim 1 and a diluent.

25. The composition of claim 24, further comprising an adjuvant.

26. A method of making an antibody, the method comprising administering to a non-human animal (a) the polypeptide of claim 1; (b) the composition of claim 24; (c) a polypeptide comprising the sequence CAHYIDGPHC (SEQ ID NO:3) or LVWKYADAG (SEQ ID NO:5); (d) a pharmaceutically acceptable composition comprising the sequence CAHYIDGPHC (SEQ ID NO:3) or LVWKYADAG (SEQ ID NO:5) and a diluent; or (e) a pharmaceutically acceptable composition comprising the sequence CAHYIDGPHC (SEQ ID NO:3) or LVWKYADAG (SEQ ID NO:5), a diluent, and an adjuvant.

27. An isolated antibody or an antigen-binding portion thereof that selectively binds to a peptide consisting of 5-20 contiguous amino acid residues that are identical to an amino acid sequence present within the second domain or the fourth domain of a receptor in the epidermal growth factor receptor (EGFR) family, with the proviso that the antibody is not the antibody designated 806 or an antibody that specifically binds a polypeptide consisting of CGADSYEMEEDGVRKC (SEQ ID NO:6).

28. An isolated antibody or an antigen-binding portion thereof, that selectively binds to a polypeptide represented by SEQ ID NO:1, SEQ ID NO:2, SEQ ID NO:3, SEQ ID NO:4, or SEQ ID NO:5.

29. The isolated antibody of claim 27, or an antigen-binding portion thereof, wherein the antibody comprises a sequence at least 80% identical to SEQ ID NO: 18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; or SEQ ID NO:39.

30. A method of inhibiting an activity of a receptor in the EGFR family, the method comprising:
providing a cell that expresses the receptor and
contacting the cell, under physiological conditions, with an amount of the isolated antibody of claim 27, or an antigen-binding fragment thereof, that is sufficient to inhibit an activity of the receptor.

31. The method of claim 30, wherein the cell is a cell maintained in culture.

32. The method of claim 30, wherein the cell is a cell in a living subject.

33. A kit comprising the isolated antibody of claim 27, or an antigen-binding fragment thereof, and instructions for diagnostic or therapeutic use.

34. A method of identifying an antibody, or an antigen binding portion thereof, that specifically binds to Domain II or Domain IV of a receptor within the EGFR family, the method comprising:
(a) providing a library of antibodies, or antigen binding portions thereof;
(b) contacting members of the library with a polypeptide comprising Domain II or Domain IV of the receptor, under conditions that allow the antibodies, or antigen binding portions thereof, to specifically bind the polypeptide; and
(c) identifying a selected antibody, or antigen-binding portion thereof, that binds to the polypeptide.

35. The method of claim 34, further comprising affinity-maturing the selected antibody, or antigen binding portion thereof, in vitro.

36. An isolated antibody, or an antigen-binding portion thereof, made by the method of claim 34.
